(19) United States
(12) Patent Application Publication
(10) Pub. No.: US 2015/0125398 A1
(43) Pub. Date: May 7, 2015

(54) MULTIMODAL IMAGING METHODS USING MESOPOROUS SILICA NANOPARTICLES

(71) Applicants: University of Iowa Research Foundation, Iowa City, IA (US); Iowa State University Research Foundation, Inc., Ames, IA (US)

(72) Inventors: Jose G Assouline, Iowa City, IA (US); Brian G. Trewyn, Arvada, CO (US); Sean K Sweeney, Iowa City, IA (US); Kapil Kandel, Webster, TX (US)

(21) Appl. No.: 14/400,518

(22) PCT Filed: Mar. 13, 2013
(86) PCT No.: PCT/US2013/031027

§ 371 Date: Nov. 11, 2014

Related U.S. Application Data
(60) Provisional application No. 61/645,712, filed on May 11, 2012.

(51) Int. Cl. A61K 49/22; A61K 49/18

(52) U.S. Cl. CPC A61K 49/221; A61K 49/22; A61K 49/1818; A61B 5/0035
USPC 424/9.32; 424/9.5

(57) ABSTRACT

The invention provides particles useful for sequential imaging or for diagnostics or therapeutics. Disclosed is a method to image diagnostic or therapeutic cells in a mammal, comprising: introducing to a mammal a composition comprising mammalian cells comprising mesoporous silica nanoparticles (MSNs) comprising a lanthanide, a fluorophore and an agent detectable by ultrasound; applying ultrasound and/or a magnetic field to the mammal and recording ultrasound and/or magnetic resonance images that include the MSNs; and detecting the presence, location or amount of the MSNs in the mammal.

### CROSS-REFERENCE TO RELATED APPLICATIONS

This application claims the benefit of the filing date of U.S. application Ser. No. 61/645,712, filed on May 11, 2012, the disclosure of which is incorporated by reference herein.

### STATEMENT OF GOVERNMENT SUPPORT

The invention was made with government support under NSF Grant No. CHE8095321. The government has certain rights in the invention.

### BACKGROUND

Contrast echocardiography is a particular niche in the larger ultrasound imaging modality, in which sound waves are transmitted through tissue and images are formed based on the timing of echoes returning to the transducer. When imaging the heart in echocardiography, contrast agents are sometimes used to highlight certain features, particularly when the patient presents with obstacles to non-contrast echocardiography, such as obesity and lung disease. Three echocardiography contrast agents are currently approved and used clinically which consist of various polymers encapsulating high molecular weight gases (Chelliah and Senior, 2009) and are typically in the 1-5 μm size range, which makes them small enough to traverse capillaries while being large enough to easily generate an echo. The gases have a dramatically slower speed of sound compared with soft tissue, giving rise to a greater contrast.

For example, Definity® is a perflutren lipid microsphere composed of octafluoropropane encapsulated in an outer lipid shell. The lipid shell is composed of hexadecanoic acid, monosodium salt and inner salt. For proper use, Definity® requires activation by warming it to room temperature and shaking for 45 seconds using a Vialmix®. Once mixed, 1 milliliter of Definity® contains about 1.2×10¹⁰ lipid microspheres and 1.1 mg octafluoropropane. After activation and intravenous injection, Definity® provides contrast enhancement of the endocardial borders during echocardiography.

Generally, nanometer-scale contrast agents are not used in ultrasound because they are much smaller than the smallest attainable spatial resolution of the ultrasound transducer. However, some namometer-scale echocardiography contrast agents are being used experimentally. Casciaro et al. (2010) tested various diameters (160, 330, and 660 nm) of silica nanobeads. Using agarose gel plates as phantoms, a signal could be observed for silica concentrations up to 0.8% and the particles could also be automatically detected using RF signal analysis (Casciaro et al., 2010).

With regard to the use of ultrasound contrast to detect injected stem cells labeled with metallic nanobeads, Bara et al. (2010) used magnetic cell separation to isolate the CD34+/CD133+ population labeled with 50 nm dextran-coated iron beads from bone marrow aspirates, then injected boluses of these cells into the myocardium of domestic pigs. Transesophageal echocardiography (TEE) was performed immediately before and after, as well as several minutes after, two injections were made. Injections of particles alone as well as unlabeled cells were made as controls. The TEE revealed good contrast originating from the dextran-iron beads (Bara et al., 2006). Mallidi et al. (2009) conjugated 50 nm gold nanoparticles to A431 cancer cells expressing high levels of epidermal growth factor receptor (EGFR) and imaged these cells using ultrasound and photoacoustic imaging. Various concentrations of labeled cells were suspended in gelatin phantoms and imaged, with contrast seen at 31,000 cells and 3.1×10⁷ nanoparticles per mL (Mallidi et al., 2005).

### SUMMARY OF THE INVENTION

A multimodal mesoporous silica nanoparticle (MSN) contrast agent is provided with unique capability for in vivo and in biomedical imaging, and also as a drug delivery of bioactive materials. Because of its size and imaging modalities, it can also be used as an effective molecular marker of stem cells or other therapeutic cells, e.g., the engraftment of stem cells in bone marrow transplant, tissue repair and/or replacement. The MSN elicits a significant signal compared to organs, tissues and cells examined with computed tomography (CT), magnetic resonance imaging (MRI), echography as well fluorescence microscopy. In one embodiment, the MSN is about 200 nm in diameter with about 3 nm to 5 nm pores that may be loaded with a drug or reagent of interest, or further functionalized, e.g., with a lanthanide. In one embodiment, the MSN is not functionalized with a chelating agent, e.g., a Gd chelating agent. For example, the surface and/or the silica framework of the MSN may be covalently functionalized with one or more of the following materials: gadolinium oxide, gold, bismuth, iron oxide, —CF₃ functional groups, and/or a fluorophore such as FITC or Texas. These materials can then be detected with one or more of the following imaging modalities: MRI, x-ray computed tomography, ultrasound/photoacoustic imaging, and/or fluorescence microscopy. The MSN gain entry into a variety of cells through nonspecific (engulfed) or specific (e.g., via extracellular binding molecules) methods. For example, the bone marrow-derived human mesenchymal stem cells may be easily tagged with the MSNs and their regenerative repair in many types of tissue, including bone, cartilage, skeletal and cardiac muscle, monitored. Furthermore, the MSN can be labeled with receptor ligands to selectively bind cells. For specific labeling, antibodies to cell surface markers may be employed e.g., to stromal cell precursor markers, such as antibodies or other ligands for CD44 (see, e.g., Yang et al., 2010), hematopoietic stem cell surface markers, such as antibodies or other ligands for CD34 and CD45 (see, e.g., Garcia-Pacheco et al., 2001), cardiac stem cell surface markers, such as antibodies or other ligands of VCAM1 (see, e.g., Uosaki et al., 2011) and SIRPA (see, e.g., Dubois et al., 2011). In one embodiment, cells may be targeted for receptor-mediated endocytosis rather than non-specific clathrin pits. Other targeting molecules include, for instance, glutamate or glutamine which have receptors with variable expression in the brain and so glutamate or glutamine labeled cells or nanoparticles could be employed to target cells or nanoparticles to the brain.

Thus, the invention provides a mesoporous silica nanoparticle (MSN) useful, for instance, as an ultrasound contrast agent. The MSN is highly porous and biocompatible, and may incorporate gadolinium oxide nanoparticles, gold, bismuth or iron oxide, and may be cofunctionalized with a biolabel and/or one or more imaging agents, e.g., a contrasting agent, and/or capped with iron, gold, or bismuth. In one embodiment, the MSNs may also be functionalized with poly(ethylene glycol) (PEG) or other organic polymers including polylactic acid (PLA), poly(lactic-co-glycolic) acid (PLGA), polyacrylate, or functionalized with amino acids or polypeptides, e.g., lysine/poly-L-lysine, glutamine, glutamate, RGD peptide (arginine-glycine-aspartate), to enhance biocompatibility. In one embodiment, the MSNs are also functionalized with trifluoropropyl moieties (—CH₂—CH₂—CF₃, abbreviated —F₃) to enhance ultrasound contrast. For example, MSN were covalently linked to gold and fluorescein (Au-FITC-MSN) or gadolinium oxide and fluorescein (Gd₂O₃-FITC-MSN). The Gd₂O₃-FITC-MSN were also functionalized with trifluoropropyl moieties (—CH₂—CH₂—CF₃), and coated in poly(ethylene glycol) (PEG) (PEG-F₃-FITC-Gd₂O₃-MSN). PEG-F₃-FITC-Gd₂O₃-MSN and Au-FITC-MSN were validated as potential ultrasound contrast agents in vitro using diluted particles in agarose phantoms as well as in fixed ex vivo hearts mounted in agarose. As discussed hereinbelow, these MSNs were observed on the VEVO® 2100 scanning system, using 30 and 40 MHz ultrasound transducers on in vitro and ex vivo phantoms. While some of the MSN described herein may be less echogenic, intracellular accumulation of a large number of the MSNs may be sufficient for detection to rapidly ensure that, for example, engraftment of a stem cell transplant has occurred.

Thus, the MSNs of the invention have use in diagnostic and therapeutic applications.

### BRIEF DESCRIPTION OF THE FIGURES

FIG. 1. Brunauer-Emmett-Teller (BET) nitrogen adsorption/desorption isotherms of FITC-Bi-MSN in (A), with Barrett-Joyner-Halenda (BJH) pore size distribution of FITC-Bi-MSN shown (inset). Powder X-ray diffraction (XRD) patterns of FITC-Bi-MSN (B), before surfactant removal (blue) and after surfactant removal (red). Gd-MSN XRD and N2-asorption analysis on the right side (C).

FIG. 2. Kinetics of FITC-MSN added to human fetal MSCs (A). Pictures were taken at the indicated times following addition of particles to the growth medium at 125 μg/mL. Particles can be seen randomly distributed after 1 hour, binding to the cell surface at 7 hours, and internalized on subsequent days. Scale bars indicate 25 μm. Plot of labeled cell growth tracked during experiment (B). Cells grew in a manner consistent with unlabeled cells, growing to confluence in about 10 days before leveling off. Plot of 2 different measures of particle uptake using two different image processing strategies (C). In one, the quantity of particles inside and outside cells was measured (blue), and in the other, the number of cells possessing at least one vesicle of particles is measured (red).

FIG. 3. 3D MR rendering of ex vivo mouse brain (A) and lung (C), with injection sites highlighted (red circles). Brain was injected with 150,000 labeled hMSCs (right hemisphere) and 50,000 labeled hMSCs (left hemisphere). Lung was injected with 50,000 labeled hMSCs. Plots of injection sites' values (in arbitrary MR units) compared with normal tissue (B and D).

FIG. 4. 3D rendering of ex vivo mouse heart into which 3 injections of 50,000 labeled hMSCs were added (A). The three injection sites were highlighted using manual segmentation methods (blue volumes, B and C), and statistical analyses performed (table).

FIG. 5. A) Schematic of poly(ethylene glycol) (PEG) functionalized, F₃-FITC-Gd₂O₃-MSN used for ultrasound image contrast enhancement. In this case, the fluorophore and Gd₂O₃ nanoparticles are covalently linked to the pore walls. Other configurations of the particle include MSN capped with gold, iron oxide, bismuth, or gadolinium oxide, which do not include the PEG or trifluoropropyl functionalization. TEM shows the mesoporous silica nanoparticle wafer, approximately 200 nm in diameter, with 5 nm pores (inset). B) Schematic of bismuth nanaparticle, F₃ and FITC functionalized MSN. In this case, the fluorophore and F₃ are covalently linked to the pore walls, and the bismuth nanoparticle is incorporated into the MSN framework.

FIG. 6. Ultrasound scanning of agars containing PEG-coated F₃-FITC-Gd₂O₃-MSN or PEG-Gd₂O₃-MSN at varying concentrations, in μg/mL (A). The grayscale values were compared with those for Definity®, a commercially available ultrasound contrast agent (B).

FIG. 7. 3D reconstructions generated from the short axis cine loop scans of an ex vivo heart injected with 200 μL Au-FITC-MSN in 20 μL saline, showing the exterior surface of the whole heart (A) and clipped plane view of the injection site (red circle) near the base of the ventricles (B).

FIG. 8. Raw 2D (A) and pseudocolor 3D rendering (B) of an ex vivo heart injected with 200 μg in 10 μL of Au-FITC-MSN particles, mounted in 1% agarose, and scanned at 30 MHz. Regions of interest (red ellipses) are exemplified by a hyperintense region and an associated shadow further away from the ultrasound transducer.

FIG. 9. Two frames of a cine loop performing real-time detection of contrast in the right ventricle of an ex vivo heart mounted in 2% agarose. Frames occur immediately before and after 200 μg F₃-FITC-MSN particles in 20 μL saline were delivered via hypodermic needle, seen as artifact (red arrow), into the right ventricular chamber (red ellipse). The chamber can be observed to become more hyperintense following the injection.

### DETAILED DESCRIPTION

Mesoporous silica nanoparticle materials have continued to attract significant interest as drug and gene delivery vehicles that can be cofunctionalized with a biolabel or contrasting agent (Giri et al., 2005; Gruenhagen et al., 2005; Hsiao et al., 2008; Kwon et al., 2008; Lai et al., 2003; Radu et al., 2004). MSNs are highly porous, biocompatible materials that have proven to have potential pharmacological applications. The MSN offer several characteristics that make it unique to other nanomaterials. These advantages include high surface area, tunable pore size and pore volume, and two independently functional surfaces, an exterior surface and interior pore surface.

The invention provides a mesoporous silicate body (particle) having one or more pores, and two or more functionalizations. The mesoporous silicate body can be a spherule having a diameter of about 40 nm to about 600 nm, about 100 nm to about 300 nm, about 150 nm to about 250 nm, about 300 nm to about 600 nm, or about 500 nm to about 4 μm. In one embodiment, the spherule has a diameter of about 50 nm to about 200 nm. The mesoporous silicate body can also be a rod having a length of about 500 nm to about 1 μm, about 400 nm to about 600 nm, or about 50 nm to about 250 nm. The rods can have various diameters, typically from about 50 nm to about 500 nm. The pores of the mesoporous silicate body can be about 1 nm to about 50 nm in diameter, e.g., less than about 30 to about 50 nm in diameter, and can be about 1 nm to about 10 nm, or about 1 nm to about 5 nm, in diameter. In one embodiment, the pores of the mesoporous silicate body are about 1 nm to about 10 nm in diameter.

For example, one type of functionalization is to immobilize molecules such as proteins, e.g., antibodies or other binding proteins or ligands, on inert surfaces of mesoporous silicate bodies, e.g., molecules useful to isolate and purify target molecules, to selectively remove contaminants, for enzyme catalysis and for chemical modification. Chemical cross-linking (conjugation) is a commonly used method for covalently immobilizing molecules, e.g., proteins, on inert surfaces. The resulting molecule may have altered properties, e.g., altered solubility, enhanced detection or other properties. For instance, the molecule that is crosslinked to the surface may aid in detection (e.g., a fluorescent molecule). With respect to cross-linking of proteins to inert surfaces, four protein chemical targets may be employed: primary amines (—NH2): this group exists at the N-terminus of each polypeptide chain and in the side chain of lysine (Lys, K) residues; carboxyls (—COOH): this group exists at the C-terminus of each polypeptide chain and in the side chains of aspartic acid (Asp, D) and glutamic acid (Glu, E); sulfhydryls (—SH): this group exists in the side chain of cysteine (Cys, C). Often, as part of a protein's secondary or tertiary structure, cysteines are joined together between their side chains via disulfide bonds (—S—S—); and carbonyls (—CHO): these aldehyde groups can be created by oxidizing carbohydrate groups in glycoproteins. For each of these protein functional group targets, there exist one to several types of reactive groups that may be used as the basis for crosslinking and for modification reagents. All of these groups are available for use with the particles described herein.

Thus, in addition to or in lieu of the functionalizationalizations described herein including a lanthanide, a fluorescent molecule and/or an ultrasound detection reagent, the particles may be functionalized with molecules that increase molecular mass, increase solubility for storage, or create a new functional group that can be targeted in a subsequent reaction step. For example, mesoporous silica nanoparticles (MSNs) may be pegylated by chemically attaching single- or branched-chain polyethylene glycol (PEG) groups to proteins, which provides for labeling, enhanced water-solubility and/or addition of inert molecular mass to proteins. In another example, MSNs may be modified with block sulfhydryls. Proteins can include sulfhydryls (e.g., the side chain of cysteine) and certain reagents are capable of reacting permanently or reversibly with sulfhydryl groups (e.g., methylmethanethiosulfonate, MMTS, and N-ethylmaleimide, NEM, respectively). These reagents add a very small “cap” on the native sulfhydryl, enabling the activity of certain enzymes to be controlled. Other modifications include the conversion of amines to a sulfhydryl-containing group to the primary amine. N-succinimidyl S-acetylthioacetate, SATA and related reagents contain an amine-reactive group and a protected sulfhydryl group. By reacting the compound and a purified protein, the side chain of lysine residues can be modified to contain a sulfhydryl group for targeting with sulfhydryl-specific crosslinkers or immobilization chemistries. The effect is also to extend the length of the side chain by several nanometers.

The above-described modifications may be to the surface and/or internal porous surfaces. In one example, proteins may be attached onto the surface using the chemical cross-linker dithiobis(succinimidyl propionate) (DSP, Product No. 22585, Pierce Biotechnology). DSP is a homobifunctional, amine-reactive cross-linker. The linkage formed between DSP and the MSN surface is very stable, exceeding the strength and stability of covalent silane bonds with glass. The disulfide linkage in DSP chemisorbs rapidly to surfaces, while the active NHS groups on either end of DSP are reactive toward primary amine groups in proteins.

Examples of molecules to be conjugated to the particles include but are not limited to low molecular weight ligands (e.g., folic acid, thiamine, dimercaptosuccinic acid), peptides (e.g., RGD, LHRD, antigenic peptides, internalization peptides), proteins (e.g., BSA, transferrin, antibodies, lectins, cytokines, fibrinogen, thrombin), polysaccharides (e.g., hyaluronic acid, chitosan, dextran, oligosaccharides, heparin), polyunsaturated fatty acids (e.g., palmitic acid, phospholipids), DNA, plasmids, siRNA, and the like.

In one embodiment, particles are modified with a carboxyl moiety on the surface to act as a universal capture molecule. For instance, N-(trimethoxysilylpropyl)ethylene diamine triacetic acid addition, either together or after TEOS is employed. Alternatively, 3-aminopropyltrimethoxysilane modification followed by carboxyl group introduction with the linker elongation, (see, e.g., Schiestel et al. (2004). The attachment of proteins may be accomplished using, e.g., 1-Ethyl-3-[3-dimethylaminopropyl]carbodiimide hydrochloride (EDC or EDAC) with N-hydroxysuccinimide (NHS) or its sulfo-derivative (sulfo-NHS), a zero-length crosslinking agent used to couple carboxyl groups to primary amines.

One example of a protein molecule for attachment to mesoporous silica nanoparticles is antibodies (immunoglobulin) of any kind (IgG, IgM, IgE, etc. . . . ). These antibodies could be directed against molecules on the surface of cells and/or organs and could be used as a marker. Then another attached marker could be used for detection (e.g., fluorescence or CT, MRI or Echo). Alternatively or in addition, these attached markers could be used therapeutically, e.g., to supply a molecule to the cells/tissue or destroy the cells/tissues they are bound to. Examples of targets and cell surface markers to which antibodies can bind are provided below.

Potential targets for functional imaging

| Cell type | Cell surface marker |
|---|---|
| Mesenchymal stem cells | CD29, CD44, CD81, CD106 |
| Hematopoietic stem cells | CD34, CD45 |
| Cardiomyocytes | VCAM1, SIRPA |
| Neurons | Receptors for glutamate, glycine, and kainite |
| Gastrointestinal tract | Colorectal Cancer: EphB4, CEACAM1, ESA, |
| | CD24, CD44, CD166 |
| | Ulcerative colitis: E-Selectin |
| | Crohn's disease: CCL20 |

The mesoporous silicate bodies may have removable caps, which can include inorganic and/or organic molecules, e.g., for imaging or drug delivery. In one embodiment, the removable cap is a particle of iron oxide, bismuth or gold, e.g., an iron oxide, bismuth oxide or a gold nanoparticle. The cap may be covalently bonded to the mesoporous silicate, optionally through a linking group. The linking group can be any suitable cleavable moiety, for example, a linking group such as 2-(propyldisulfanyl)ethylamine, a urea containing group, or a linking group with groups susceptible to oxidation, e.g., by a reducing agent such as DTT, glutathione, cysteine, or dihydrolipoic acid, a linking group such as X—CH₂CH₂CH₂SSCH₂CH₂NH—C(═O)CH₂—Y, wherein X is a silicon atom of the mesoporous silicate and Y is a cap for the pore. The cap of the mesoporous silicate may include an organic polymer. For example, the cap may include a poly(amidoamine), a polypeptide, or an oligonucleotide. The cap may also be a hyper-branched polymer. One example of a hyper-branched polymer is a dendrimer. Dendrimer caps can be anionic, neutral or cationic dendrimers. The caps may also be a biodegradable polymer, such as a poly(amidoamine).

The particles of the invention are useful in a variety of methods, including imaging, with or without the use of administered cells, and therapeutic drug/cell delivery. In one embodiment, the MSNs of the invention include a lanthanide such as gadolinium. Gadolinium has gained popularity as an MRI contrast agent because, like iron oxide, it affects large changes in the local magnetic fields where it is present. By virtue of the fact that it has 7 unpaired electrons in its outer shell, it interacts very efficiently with surrounding protons. If the same specimen is scanned at two different echo times, the changes in field effects between the two scans is larger relative to the differences between background materials. Therefore, a simple subtraction of one image at one echo time from the other further enhances the tracing of the material.

### Magnetic Resonance Imaging (MRI)

Magnetic resonance imaging uses small variations in the magnetic field arising from differing proton spin densities ρ(x,y) in tissue to generate its images. Briefly, the grayscale value at each pixel in a slice of an MR image is the 2-dimensional inverse Fourier transform of that slice's k-space, or frequency domain s(t). The radio frequency (RF) data encoded in the frequency domain is collected when small perturbations are made in the larger magnetic field of the MR scanner using smaller gradient coils that vary over time as Gₓ(t) and G_{y}(t). The general equations describing the signal are

$$s ( t ) = {\int }_{- ∞}^{∞} {\int }_{- ∞}^{∞} ρ ( x , y ) {e}^{- j2π [ {k}_{x} ( t ) x + {k}_{y} ( t ) y ]} d x d y , \\ where$$

$${k}_{x} ( t ) = {\int }_{0}^{t} \frac{γ}{2 π} {G}_{x} ( τ ) d τ , \\ and$$

$${k}_{y} ( t ) = {\int }_{0}^{t} \frac{γ}{2 π} {G}_{y} ( τ ) d τ .$$

The MR signal of a specific tissue can also be described by its parameters (T₁, T₂, T₂* relaxation times) and the parameters of the scan (repetition time TR, echo time TE, and/or flip angle α). After a material is magnetized with a certain flip angle α, the magnetic field in the longitudinal axis M_{z} decays (relaxes) with time constant T₁, and varying the time between pulses (repetition time T_{R}), tissue with different T₁ relaxation times show up with different levels of intensity in the reconstructed image. This is known as a T₁-weighted image. In a T₂-weighted image, the echo time T_{E} (the time between the pulse and the midpoint of signal readout) is used to generate differing intensities between tissues of different T₂/T₂* relaxation times (the time constant of relaxation or “de-phasing” in the transverse plane, or M_{xy}) (Liang and Lauterbur, 2000).

Besides varying T_{R}/T_{E} on the scanner to achieve the desired contrast, additional contrast can be achieved by using one of several contrast agents. MRI contrast agents are ferromagnetic, paramagnetic, or superparamagnetic materials which interact with the protons present in the surrounding medium, thereby altering the apparent T₁ or T₂ relaxation time. The effect of contrast agents on the observed T₁ or T₂ value is given by the equation

$$\frac{1}{{T}_{obs}} = \frac{1}{{T}_{tissue}} + r [ contrast ] ,$$

where T_{obs} is the observed T₁ or T₂ value, Tₜᵢₛₛᵤₑ is the actual T₁ or T₂ relaxation time of the tissue being scanned, r is the r₁ or r₂ relaxivity of the contrast agent, measured in s⁻¹·mM⁻¹, and [contrast] is the molar concentration of the contrast agent (Lauffer, 1987).

Most ferromagnetic contrast agents involve the use of superparamagnetic iron oxide (SPIO), which perturbs the tissue's local magnetic field, causing a change in T₂ or T₂* relaxation time.

In MRI, the signal-to-noise ratio (SNR) is proportional to the magnetic field, the voxel size, and the square root of total scan time. Compared to the above scan parameters, the magnetic field of a clinical scanner is reduced by a factor of 3, the voxel size is increased by a factor of about 2, and the scan time is reduced by as much as 8 times (from up to 4 hours to about a half hour). Therefore, the reduction in signal can be estimated as 2/(3\sqrt{8}, or about 4.2 times smaller. This can be approximately balanced out by increasing the number of labeled cells from the 5×10⁴ that was detected in the above scans to about 2×10⁵. For both of these estimations, the actual thresholds are likely to vary from these estimates, and for different tissues, and can and should be confirmed through testing of scaled-up phantoms in the clinical scanners at a time when clinical testing is more imminent.

### X-Ray Computed Tomography (CT)

Unlike MRI in which the contrast is derived from magnetic properties of tissue, CT images are essentially based on the density of the tissue in the path of the x-rays. In summary, x-ray photons at a known energy are projected towards the patient and detected on the other side. The simplified equation of intensity of the photons striking the detector is given by the relationship

I(x)=I₀e^{-μx},

where I₀ is the initial intensity and p represents the attenuation coefficient of the material (a function primarily of tissue density). In order to generate a multislice CT image, this principle is expanded to a 2 dimensional detector which can be rotated around the body. Many 2-D projections are compiled into a 3-D image according to the equation

$${I}^{θ k} = {I}_{0} {e}^{- {Σ}_{ij} {w}_{ij}^{θ k} {μ}_{ij}} ,$$

where I^{θk} is the intensity data for detector position k and angle θ, wᵢⱼ is a weighting value for position (i,j) on the detector at position k and angle θ, and μᵢⱼ is the attenuation of the material at position (i,j) (Webster and Clark, 1998).

In x-ray CT, contrast agents are effective if they have an ability to greatly change the x-ray opacity of the tissue of interest. Therefore most of the early contrast agents were based on heavy elements such as iodine and barium. Because of toxicity concerns, these agents have evolved over time, and other contrast agents based on electron-dense metals have also been studied, and are well reviewed by Yu/Watson. Of the heavy metal contrast agents, those based on gold, bismuth and gadolinium appear to be the most studied (Yu et al., 1999).

Multimodal particles are particularly useful to enhance signals in multiple scanning modalities. For example, a bolus of particles or cells labeled with MSNs is introduced to a host organism, e.g., a mammal, in a single dose and the recipient/subject is sequentially subjected to different scanning modalities (e.g., Echo, MRI and/or CT). Some of the faster/less time consuming scans (e.g., Photoacoustic) may be used as a guide for more comprehensive scans (e.g., MRI and/or CT). Resulting image analysis for the various analyses can then be compared for more discriminated evaluation. The MSN of the invention are quite useful in such sequential procedures as they are very stable over time unlike traditional contrast agents which are for only one modality and are active for only short periods of time after reconstitutions (for instance. for minutes or a couple of hours (Englebrecht et al., 1996).

### Ultrasound

Ultrasound is perhaps the fastest and safest way to obtain in situ images, as it requires only a few seconds of preparation with ultrasound gel and produces no ionizing radiation. The drawback is that the spatial resolution does not approach what is possible in CT or MRI at this time. In this modality, a piezoelectric transducer produces sound at high frequencies (typically between 2 and 15 MHz for clinical applications and up to 40 MHz or more for research applications) and generates an image based on the timing of echoes returning to the transducer. Echoes are generated when the propagated sound wave strikes an interface between volumes with differing acoustic impedances (Z) and part of the sound wave reflects back to the transducer. Acoustic impedance is defined as

Z=ρc,

where ρ is the density and c is the speed of sound in the tissue⁷⁵. At the interface between two tissues, the reflectance coefficient (R) describes the fraction of sound energy that will be reflected back to the transducer. The remaining fraction continues propagating deeper into the tissue where it may strike another interface. R is related to the acoustic impedances of the two tissues at the interface (Z₁ and Z₂) according to the equation

$$R = {[ \frac{{Z}_{1} - {Z}_{2}}{{Z}_{1} + {Z}_{2}} ]}^{2} .$$

These principles are applied to the generation of ultrasound images. In A-mode imaging, one transducer is used to plot all the tissue boundaries along one axis as a function of time. One application of A-mode imaging is tracking opening and closing of heart valves or movement of a ventricle during the heart cycle in echocardiography. In B-mode imaging, an array of transducers is coordinated to form a 2D image. This may be the most common way ultrasound is used, and includes fetal sonography among other applications. Newer ultrasound systems are capable of Doppler mode, in which frequency shifts in the sound wave are used to calculate blood flow through arteries, and even 3D ultrasound, in which the transducers are swept across many 2-D fields in rapid succession to generate a 3-dimensional image (Webster and Clark, 1998).

### Exemplary Imaging/Image Processing Parameters

### MRI

Eight 500 μL Eppendorf tubes were filled with 300 μL of varying concentrations of nanoparticles (1 Eu—Gd₂O₃: 150 and 500 μg/mL; 0.5% Eu—Gd₂O₃: 50, 150 and 500 μg/mL) and suspended in a highly viscous suspension of type I collagen derived from rat tail tendon (thhis would prevent formation of a pellet during the MRI scan). In addition, a tube containing collagen only was added as a negative control. Tubes were placed in the 4.7 Tesla Varian® MR scanner and scanned to determine the r₂ relaxivity through a sequence of T₂-weighted scans (relaxation time T_{R}=35 ms, echo time T_{E}=4, 6, 8, 10, 12, 14, 16, 18, and 20 ms). In addition, pairs of T₂* gradient echo scans (T_{R}=35 ms and T_{E}=6 and 14 ms, 256 slices and a voxel size of 148 μm per side) were performed.

To calculate the r₂ relaxivity of the gadolinium oxide particles, the program “MRI Analysis Calculator” was downloaded from the ImageJ website (http://rsbweb.nih.gov/ij/plugins/mri-analysis.html). This program requires as input an image stack containing the same slice of data at each of the different echo times. It then calculates the T₂ value at each pixel in the slice using a Simplex best-fit algorithm to solve the following equation for T₂:

$${S}_{n} = {S}_{0} {e}^{( - {T}_{{E}_{n}} / {T}_{2} )} ,$$

where Sₙ is the signal value at the pixel at each echo time T_{En} and S₀ is the initial magnetic field (a constant).

After obtaining the T₂*-weighted scans, post-processing was done in both ImageJ and MIPAV. In ImageJ, the images from the 2 different scans were subtracted, and the difference saved as a third image. This difference image was also put through the automated background subtraction algorithm in ImageJ, with a radius of 50 pixels, to smooth the background noise. In MIPAV, volumes of interest (VOIs) of each of the tubes in both the T_{E}=6 ms and the difference image were selected by manual segmentation. Additional VOIs were selected for PBS controls. VOI volumes and average MR intensities were obtained and saved to a separate file for further analysis.

The standard deviations of the average intensities, when measuring the entire volume of the tube, were similar to those of other scans, so it was deemed that the dispersion was maintained reasonably well for the duration of the scan. When the difference-subtracted images were normalized to collagen and measurements made, the resulting plots reflected the scans. The signal intensity in europium-doped gadolinium was about 30% higher than collagen at 50 μg/mL, and increased to about 50% higher at 500 μg/mL.

A new T₂-weighted scan at several echo times (4, 6, 8, 10, 12, 14, 16, 18, and 20 ms) was performed so that the r₂ relaxivity value could be calculated and compared with other available particles. Using MATLAB, the original MR images for the tubes were split into individual slices which were reorganized so that each slice contained all the echo times stacked together. One by one, these were processed using the ImageJ plugin “MRI Analysis Calculator”. The calculated T₂ times were reassembled into a single stack so that MIPAV volume-of-interest (VOI) tools could be used to isolate the different concentrations within the scan. The mean T₂ value was calculated for each scan, and a plot of 1/T₂ vs. concentration was made. The plot shows the equation of the best fit line, used to determine the r₂ relaxivity value for the particles, which was 3.6 s⁻¹·mM⁻¹, and with a strong R-squared value of 0.98.

For MRI using MSN particles, cells are prepared according to recognized protocols for isolation and labeling, using nanoparticle capped (iron oxide, gold, or bismuth), FITC-loaded MSN particles (Fe/FITC-MSN, Au/FITC-MSN and Bi/FITC-MSN or Gd₂O₃/FITC MSN) at a concentration of 125 μg/mL in the growth media. One day after labeling and immediately prior to the experiments, the cells are loosened from the surface of their culture dish using TRYPLEexpress® (GIBCO), suspended in a small volume of phosphate buffered saline (PBS, GIBCO), sampled and counted using Trypan Blue exclusion dye and a hemacytometer. A mouse is then given an intraperitoneal injection of 0.1 mL heparin and anesthetized in a chamber of isofluorane until non-responsive to paw pinching with forceps.

For ex vivo MR imaging, the chest cavity is opened and the inferior vena cava (IVC) is severed. A gravity-fed apparatus containing normal saline with a 22 gauge needle is inserted into the right ventricle of the mouse to clear the blood from the vasculature. Both fluids are set on a shelf approximately 1.5 meters above the benchtop in order to deliver the fluids at a hydrostatic pressure of about 110 mmHg, or roughly the systolic pressure of a normal mouse. After the blood draining from the IVC runs clear, the apparatus is switched to deliver 4% paraformaldehyde. Perfusion fixation is continued until the mouse's tail curled and then went straight, a sign of muscle fibers cross linking (about 10 minutes of flow). Injections of quantities of Fe/FITC-MSN labeled cells are made into the tissues as noted, as are PBS sham injections and needle sticks only as controls. T2*-weighted pulse echo sequences are used for MR imaging.

After perfusion fixation of a mouse, the brain and lungs were dissected and injected with a number of Fe/FITC-MSN-labeled stem cells. Injections of 150,000 and 50,000 cells were made into each hemisphere of the brain, and a 50,000 cell injection was made in the lung through the pleura. The organs were stored in 15 mL centrifuge tubes with 4% paraformaldehyde and scanned in the 4.7 Tesla Varian® small animal scanner. After opening the images in MIPAV, each injection site could be observed in the 3D reconstructions of each organ. The volumes of interest were selected and measurements were made: total volume in voxels and in mm³, and average and standard deviation of the intensity value (in arbitrary units). Control volumes of interest were selected as well from normal tissue away from the injection sites. Statistical comparison of two means was performed on the data, and significance (p<0.05) was observed. In the case of the brain, comparison of the injection site to the nearby ventricles did not show a significant difference.

In the case of the heart, 3 injections of 50,000 cells each were made in the same heart, and their intensity value averaged. This average was compared to both normal heart tissue as well as to air bubbles which were trapped in the centrifuge tube, because to the naked eye, these had a similar hypointense value as the injections of cells. In both comparisons, significance was observed.

### CT

For micro-CT imaging, a similar method is used; however, prior to opening the chest cavity, the trachea is exposed, partially cut, and cannulated with a flexible 22 gauge Luer-lok cannula. Through the cannula, 1.7×10⁶ cells labeled with one of the above mentioned MSN particles, e.g., Au/FITC-MSN, are delivered to one of the lungs. The lungs are then inflated by connecting the cannula to a source of air pressure for the remainder of the perfusion fixation. The heart/lungs are dissected out as one unit, still under air pressure through the trachea, and dried in a drying oven for several days. Scans are performed at varying voltages and currents.

For both CT and MRI, the freeware medical image processing program MIPAV is used for image analysis. The isolevel selection tool is used to manually segment volumes of interest (VOIs): in MR heart imaging, the injection sites as well as control volumes for myocardium and paraformaldehyde, and in CT lung imaging, the terminal bronchioles containing labeled cells as well as an unlabeled region in the contralateral lung. For each VOI, MIPAV calculates the mean and standard deviation of intensity value and number of voxels, and these figures are used for pairwise statistical analysis using the t-test for comparison of two means with independent samples and unequal variances:

$$t = \frac{( {\overline{x}}_{1} - {\overline{x}}_{2} ) - ( {μ}_{1} - {μ}_{2} )}{\sqrt{( {s}_{1}^{2} / {n}_{1} ) + ( {s}_{2}^{2} / {n}_{2} )}} ; v = \frac{{[ \frac{{s}_{1}^{2}}{{n}_{1}} + \frac{{s}_{2}^{2}}{{n}_{2}} ]}^{2}}{[ \frac{{( {s}_{1}^{2} / {n}_{1} )}^{2}}{{n}_{1} - 1} + \frac{{( {s}_{2}^{2} / {n}_{2} )}^{2}}{{n}_{2} - 1} ]}$$

where n₁ and n₂ are the number of voxels in each VOI, s₁² and s₂² are their respective standard deviations, and are their means and ν is the degrees of freedom used in reference to the statistical lookup table.

### Exemplary Cells for Administration

Cells within the scope of the invention, e.g., those which are labeled with the MSNs described herein, include but are not limited to bone marrow-derived cells, e.g., mesenchymal cells and stromal cells, smooth muscle cells, fibroblasts, SP cells, pluripotent cells or totipotent cells, e.g., teratoma cells, hematopoietic stem cells, for instance, cells from cord blood and isolated CD34⁺ cells, multipotent adult progenitor cells, adult stem cells, embyronic stem cells, skeletal muscle derived cells, for instance, skeletal muscle cells and skeletal myoblasts, cardiac derived cells, myocytes, e.g., ventricular myocytes, atrial myocytes, SA nodal myocytes, AV nodal myocytes, and Purkinje cells. Thus, the cells include embryonic, fetal, pediatric, or adult cells or tissues, including but not limited to, stem cells and precursors (progenitor) cells. For example, the cells can be myocardial cells, bone marrow cells, hematopoietic cells, lymphocytes, leukocytes, granulocytes, hepatocytes, monocytes, macrophages, fibroblasts, neural cells, mesenchymal stem cells, beta-islet cells, and combinations thereof, or cells capable of differentiating into those cells. In one embodiment, the cells are autologous cells, however, non-autologous cells, e.g., xenogeneic or allogeneic cells, may also be employed.

Routes of Administration of MSNs or Cells Labeled with MSNs

The compositions of the present invention may be administered by any means known in the art. For example, the compositions are suitable for parenteral administration, for instance by intramuscular, subcutaneous or intravenous routes. The compositions of the invention may also be administered subcutaneously, into vascular spaces, or into joints, e.g., intraarticular injection. The local delivery of the compositions can also be by a variety of techniques. Examples of delivery vehicles include catheters, such as an infusion or indwelling catheter, a needle or other device for injection, implantable devices, or site specific carriers.

The compositions suitable for injection or infusion may include sterile aqueous solutions or dispersions or sterile powders comprising the MSNs which are adapted for the extemporaneous preparation of sterile injectable or infusible solutions or dispersions. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For example, for engraftment, cells are labeled with the particles and then implanted into a recipient (e.g., human or other primate, or other mammal). Alternatively, the nanoparticles are injected as a bolus directly into an organ of interest. Parenteral injections are also envisioned and are warranted for certain applications. Catheter based delivery of the nanoparticles or cells may be employed, e.g., for delivery within the brain with minimal trauma to surrounding structures and so as to avoid to critical cerebral structures, yet allowing for delivery to deep zones. Translumenal catheter based approaches are also envisioned, e.g., for treatment of stroke, chronic neurological diseases or cerebrovascular diseases. Catheters may also be used to deliver nanoparticles or cells, for instance, progenitor cells, having nanoparticles intramyocardially or intravascularly, e.g., via an intracoronary approach. Monitoring of the nanoparticles or cells may be accomplished by photoacoustic (ultrasound), MRI and/or CT imaging.

In addition, microrobots and nanorobots may be employed, e.g., for repairs that are currently being performed laparoscopically. In this approach, nanoparticles are introduced (e.g., via a microrobot or nanorobot), or injected into areas of interests and are activated by creating intermittent acoustic/electric or magnetic fields.

The invention will be further described by the following non-limiting examples.

### Example I

### Synthesis of Fe and Au NP Capped MSNs

The Fe₃O₄ NP and Au NP capped-MSN materials were initially made by the following procedure: First, 1.2 mg fluorescein isothiocyanate was stirred for 20 minutes at room temperature with 10 μL 3-aminopropyltrimethoxysilane (APTMS) in 400 μL anhydrous THF. Next, n-cetyltrimethylammonium bromide (CTAB, 1.0 g, 2.7×10⁻³ mol) was dissolved in 480 mL nanopure water (353 K), made basic with 3.5 mL 2.0 M NaOH. Tetraethyl orthosilicate (TEOS) (5.0 mL, 2.6×10⁻³ mol) was first introduced dropwise, followed by the dropwise addition of the FITC-APTMS/DMF solution. The mixture was stirred for 2 hours at 353 K to give rise to an orange precipitate (as-synthesized FITC-MSN). The solid product was filtered, washed with deionized water and methanol, and dried under vacuum. To remove the surfactant template (CTAB), the as-synthesized FITC-MSN (1.0 g) was refluxed for 18 hours in a solution of 1 mL HCl (37.4%) and 100 mL of methanol, followed by washing with water and methanol.

### Synthesis of Gadolinium Oxide Colloid

The gadolinium oxide colloid was obtained following the previously reported synthesis (Bridot et al., 2007). Gadolinium (III) chloride hexahydrate (11.53 g) was dissolved in 200 mL of diethylene glycol at 60° C. overnight under vigorous stirring. Aqueous sodium hydroxide (7.5 mL, 3M) was added and the solution was heated at 140° C. for 1 hour and then at 180° C. for 4 hours. The obtained transparent colloid of gadolinium oxide nanoparticles was stored at room temperature.

### Synthesis of Gadolinium Oxide Functionalized Mesoporous Silica Nanoparticles

Cetyltrimethylammonium bromide (CTAB), (CH₃(CH₂)₁₅N(CH₃)₃Br) (1.0 g, 2.745 mmol) was dissolved in nanopure water (480 g, 26.67 mol), followed by the addition of NaOH solution (2.0 M, 3.5 mL, 7.0 mmol). The mixture was heated to 80° C. for one hour. To this clear solution, tetraethoxysilane (4.7 g, 22.56 mmol) was added drop wise, followed by immediate addition of gadolinium oxide colloid (1 mL). The reaction was stirred vigorously at 80° C. for 2 hours and then the solution was filtered to yield white gadolinium oxide functionalized mesoporous silica nanoparticles. The as-synthesized material was washed with copious amount of water and methanol and then dried under vacuum. The CTAB surfactant was removed by Soxhlet extraction with methanol for 24 hours and then dried under vacuum to obtain Gd-MSN.

### Synthesis of Fluorescein Isothiocyanate Functionalized Gadolinium Oxide Mesoporous Silica Nanoparticles (FITC-Gd-MSN)

FITC (5 mg, 0.0128 mmol) was reacted with (3-aminopropyl)trimethoxysilane (2.2345 μL, 0.0128 mmol) in DMSO for 2 hours. FITC-Gd-MSN was prepared by grafting 0.05 mL of resulting product on Gd-MSN (100 mg) in toluene under reflux for 24 hours. The resulting solution was filtered and the obtained yellow solid was washed with copious amount of methanol and then dried under vacuum.

### Synthesis of Polyethylene Glycol Functionalized Gadolinium Oxide Mesoporous Silica Nanoparticles (PEG-Gd-MSN)

PEG-GD-MSN was prepared by grafting 2-[Methoxy(polyethyleneoxy)propyl]trimethoxysilane (0.2 mmol) on Gd-MSN (100 mg) in toluene under reflux for 24 hours. The resulting solution was filtered and the obtained white solid was washed with copious amount of methanol and then dried under vacuum.

### Synthesis of (3,3,3-trifluoropropyl)trimethoxysilane Functionalized Mesoporous Silica Nanoparticles (TFP-MSN)

The mixture of cetyltrimethylammoniumbromide surfactant (CTAB), (CH₃(CH₂)₁₅N(CH₃)₃Br) (1.0 g, 2.745 mmol), 2.0 M of NaOH (aq) (3.5 mL, 7.0 mmol) and H₂O (480 g, 26.67 mol) was heated to 80° C. for an hour. To this clear solution, tetraethoxysilane (4.7 g, 22.56 mmol) was slowly added and then (3,3,3-trifluoropropyl)trimethoxysilane (1.0 mL, 5.21 mmol) was added rapidly via injection. The reaction was stirred vigorously at 80° C. for 2 hours and then the solution was filtered to yield white TFP-MSN solid. The as-synthesized material was washed with copious amount of water and methanol and then dried under vacuum. The CTAB surfactant was removed by soxhlet extraction with methanol for 24 hours and the resulting surfactant removed solid was dried under vacuum.

### Synthesis of Fluorescein Isothiocyanate Functionalized TFP-MSN (FITC-TFP-MSN)

FITC (5 mg, 0.0128 mmol) was reacted with (3-aminopropyl)trimethoxysilane (2.2345 μL, 0.0128 mmol) in DMSO for 2 hours. FITC-TFP-MSN was prepared by grafting 0.05 mL of resulting product on TFP-MSN (100 mg) in toluene under reflux for 24 hours. The resulting solution was filtered and the obtained yellow solid was washed with copious amount of methanol and then dried under vacuum.

### Synthesis of (3,3,3-trifluoropropyl)trimethoxysilane Functionalized FITC labeled Gadolinium Oxide Mesoporous Silica Nanoparticles (TFP-FITC-Gd-MSN)

TFP-FITC-Gd-MSN was prepared by grafting (3,3,3-trifluoropropyl)trimethoxysilane (0.2 mmol) on FITC-Gd-MSN (100 mg) in toluene under reflux for 24 hours. The resulting solution was filtered and the obtained solid was washed with copious amount of methanol and then dried under vacuum.

Fluorescein and bismuth labelled mesoporous silica nanoparticle (FITC-Bi-MSN) materials were synthesized in a similar fashion with the exception of the addition of the dropwise addition of a solution of 1.0 g Bi(NO₃)₃.5H₂O, dissolved in 5.0 mL of acidified nanopure water after the dropwise addition of the FITC labelled ligand.

Gold nanoparticle (Au NP) and iron oxide nanoparticle (Fe₃O₄ NP) caps were synthesized and attached to the MSN via the same procedure as previously described in Giri et al. (2005) and Tourney et al. (2007).

### Material Characterization

Three different types of mesoporous silica nanoparticle materials were successfully synthesized and characterized by standard techniques. The three different types of MSN material are FITC-labeled, bismuth containing MSN (FITC-Bi-MSN), FITC-labeled, gadolinium nanoparticle impregnated MSN (FITC-GdNP-MSN), and labeled gold nanoparticle capped MSN (FITC-AuNP-MSN).

The structures and surface properties of the MSN were analyzed utilizing a series of different material characterization techniques, including transmission electron microscopy (TEM), energy dispersive spectroscopy (EDS), powder X-ray diffraction (XRD), nitrogen sorption, and zeta potential analysis.

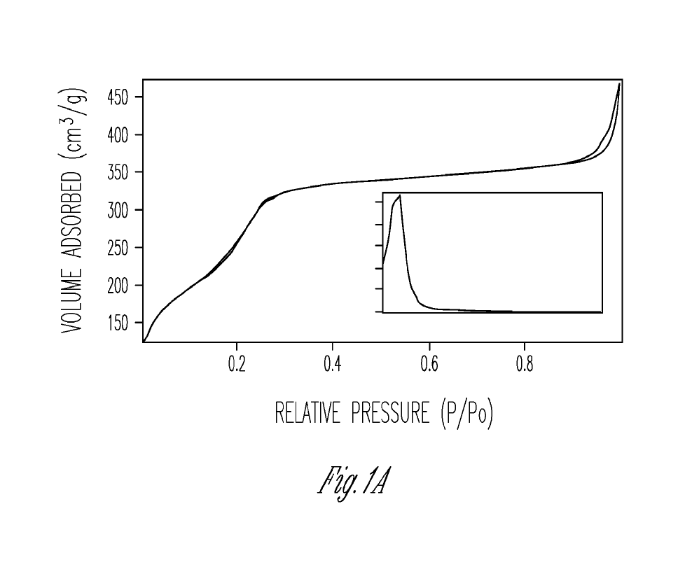

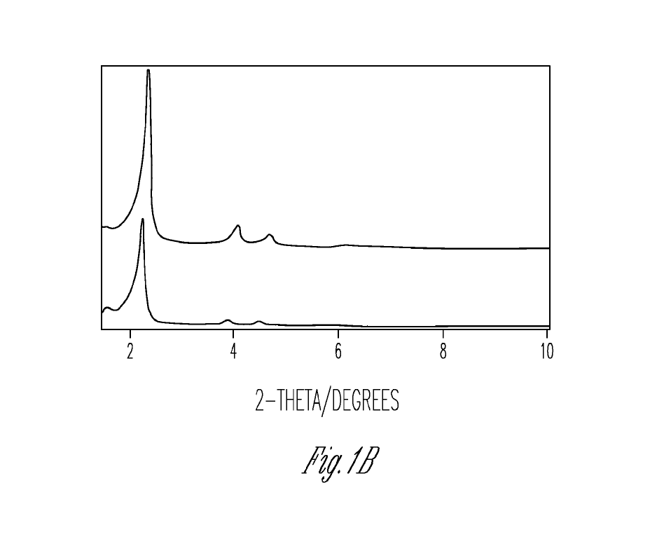

The materials were characterized by X-ray diffraction in a Rigaku Ultima IV diffractometer, nitrogen sorption analysis in a Micromeritics ASAP 2020 surface area and porosity analyzer using the Brunauer-Emmett-Teller equation to calculate surface area and pore volume and the Barrett-Joyner-Halenda equation to calculate the pore size distribution (FIG. 1). The materials were visualized by transmission electron microscopy (TEM) by supporting samples on copper grids in a Tecnai G2 F20 microscope operating at 200 kV.

Powder XRD analysis confirmed hexagonally arranged mesopores in the diffraction pattern of FITC-Bi-MSN as evident by the intense d₁₀₀, and well resolved d₁₁₀ and d₂₀₀ peaks characteristic for MSN. Nitrogen sorption analysis of the FITC-Bi-MSN exhibited a Type-IV isotherm, typical of mesoporous materials with a BET surface area of 710 m²g⁻¹. The average pore diameter for FITC-Bi-MSN by BJH calculation is 24 Å.

### MSN Uptake Kinetics

A stock solution of each species of MSN was created by suspending 5 mg powdered MSN in 500 μL PBS. The suspensions were sonicated for 30 minutes in a sonicating water bath and a series of dilutions from 10-human bone marrow derived mesenchymal stem cells in each well of a 24 well tissue culture plate. After up to a week of incubation, the maximum dose that did not cause excessive cell death was found to be 125 μg/mL. From that point forward, this was the dosage most commonly used. To study uptake over time, 100 μg MSNs were added to each of the wells of mesenchymal stem cells seeded previously. To view uptake of the particles, the 6 well plate containing FITC-MSN particles was imaged using the Olympus® IX70 fluorescence microscope with green 494 nm filter and with an attached DP70 digital camera and software. Images were obtained at 1 hour, 6 hours, and 26 hours following addition of the particles. After 27 hours, particles that were not engulfed were rinsed from the culture using D-PBS, and more images taken on subsequent days.

Image analysis was done using a MATLAB program. Briefly, the images were normalized to use the entire range of green pixel values from 0-255 with no saturation, then the built-in MATLAB edge detection function was used to isolate single cells. The extracellular area was masked out, and bright green pixels within cells above a user-defined threshold were counted, and these numbers were used to estimate the volume of MSN particles internalized over time.

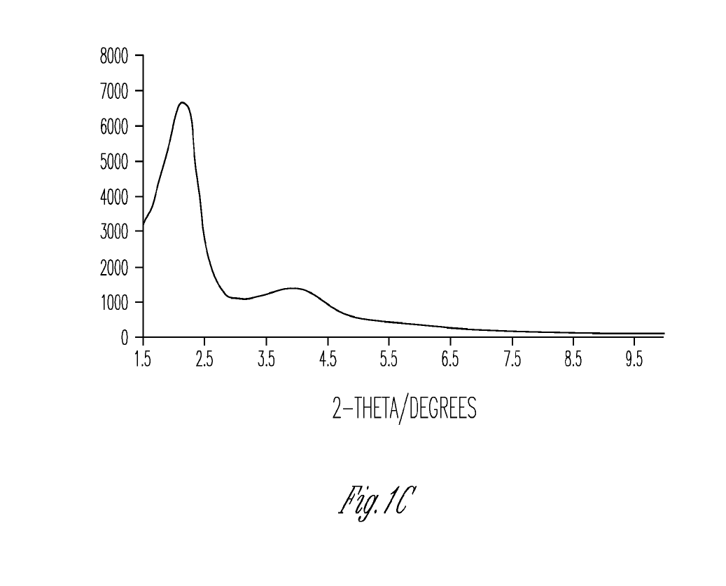

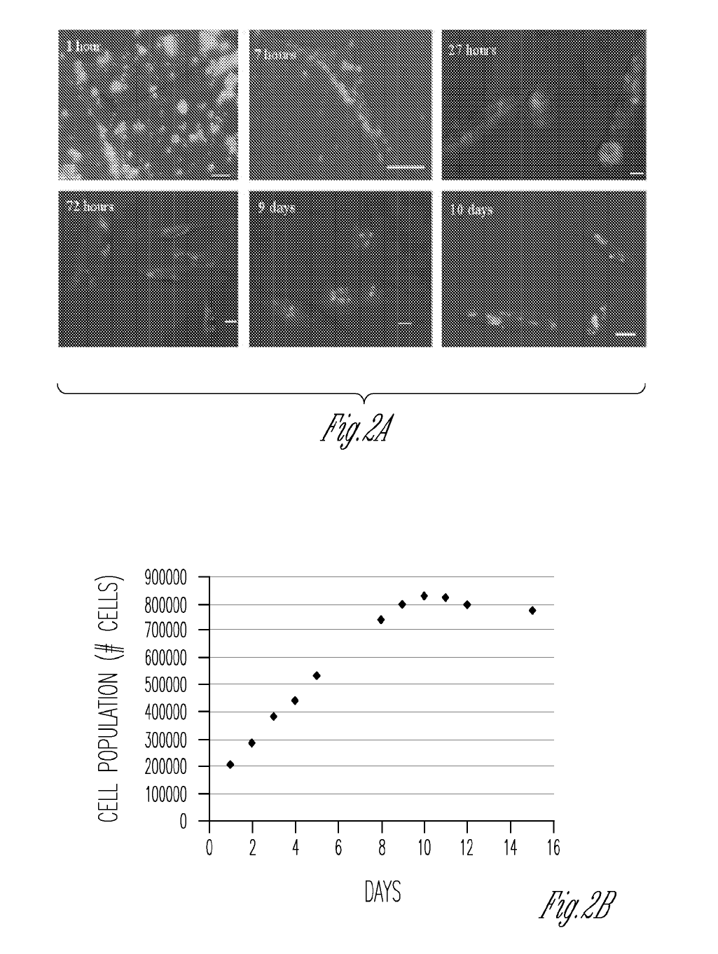

Early binding of FITC-MSN particles to cell membranes was observed after one hour, with further binding and early endocytosis by 7 hours (FIG. 2). At 27 hours, most of the particles appeared to be in the outer regions of cell cytoplasm, and few particles remained outside the cells. At this point the cells were rinsed with phosphate buffered saline to confirm that the particles were indeed bound to the cells, rather than settled loosely on top of them. As the days progressed, the particles were seen to homogenize into fewer, larger compartments and migrate towards the cell nuclei, indicating that they are being compartmentalized, most likely in vesicles. A few dividing cells were observed at 96 hours, showing the equal cytoplasmic division of particles between daughter cells. The same patterns of internalization, compartmentalization and division appeared with the iron oxide- and bismuth-capped MSN particles. It was also observed that a few cells that took up a disproportionately large amount of particles were found to be apoptotic.

### Ex Vivo Imaging

Cells were prepared according to the above protocols for isolation and labeling, this time using nanoparticle capped (iron oxide, gold, or bismuth), FITC-loaded MSN particles (Fe/FITC-MSN, Au/FITC-MSN and Bi/FITC-MSN) at a concentration of 125 μg/mL in the growth media. One day after labeling and immediately prior to the experiments, the cells were loosened from the surface of their culture dish using TRYPLEexpress® (GIBCO), suspended in a small volume of phosphate buffered saline (PBS, GIBCO), sampled and counted using Trypan Blue exclusion dye and a hemacytometer. A mouse was then given an intraperitoneal injection of 0.1 mL heparin and anesthetized in a chamber of isoflurane until non-responsive to paw pinching with forceps.

For ex vivo MR imaging of mouse tissue, the chest cavity was opened, and the inferior vena cava (IVC) was severed. A gravity-fed apparatus containing normal saline with a 22-gauge needle was inserted into the right ventricle of the mouse to clear the blood from the vasculature. Both fluids were set on a shelf approximately 1.5 meters above the benchtop in order to deliver the fluids at a hydrostatic pressure of about 110 mmHg, or roughly the systolic pressure of a normal mouse. After the blood draining from the IVC ran clear, the apparatus was switched to deliver 4% paraformaldehyde. Perfusion fixation was continued until the mouse's tail curled and then went straight, a sign of muscle fibers cross linking (about 10 minutes of flow). Injections of quantities of Fe/FITC-MSN labeled cells were made into the tissues as noted, as were PBS sham injections and needle sticks only as controls. T2*-weighted pulse echo sequences were used for MR imaging.

For ultrasound imaging, a 1% agarose in PBS solution was made, and held at 50° C. while the other materials were prepared. Gold-capped, FITC-loaded MSN nanoparticles were sonicated for 5-10 seconds, and 20 μL at a concentration of 10 mg/mL was injected into the wall of the left ventricle of a 16 week fetal heart. No visible regurgitation of the injection out of the needle hole could be observed. Ten mL of agarose was poured into a 25 mL beaker onto which the ex vivo heart would be mounted. When it was solid enough to support the weight of the heart, it was mounted in such a way that the ventral heart would be parallel to the flat surface of the agar. An additional 10 mL molten agar was used to cover the heart and it was allowed to set. Next, 5 mL molten agar was poured into each of 7 60 mm dishes. The nanoparticles were added to the agars to make concentrations of 0, 25, 50, and 200 μg/mL, and the stock solution of 10 mg/mL was scanned within its Eppendorf tube as well.

At the ultrasound scanner, the 30 MHz transducer was used. Each prepared agar was carefully removed from its Petri dish, coated with a layer of ultrasound gel, and scanned. For comparison studies, the gain was held constant at 28 dB; all other scanning parameters were kept constant as well. For the heart, several cine loops in both the short axis and long axis were made which ran the length of the entire organ.

For all imaging modalities, the freeware medical image processing program MIPAV was used for image analysis. The isolevel selection tool was used to manually segment volumes of interest (VOIs): in MR heart imaging, the injection sites as well as control volumes for myocardium and paraformaldehyde, and in CT lung imaging, the terminal bronchioles containing labeled cells as well as an unlabeled region in the contralateral lung. For each VOI, MIPAV calculated the mean and standard deviation of intensity value and number of voxels, and these figures were used for pairwise statistical analysis using the t-test for comparison of two means with independent samples and unequal variances.

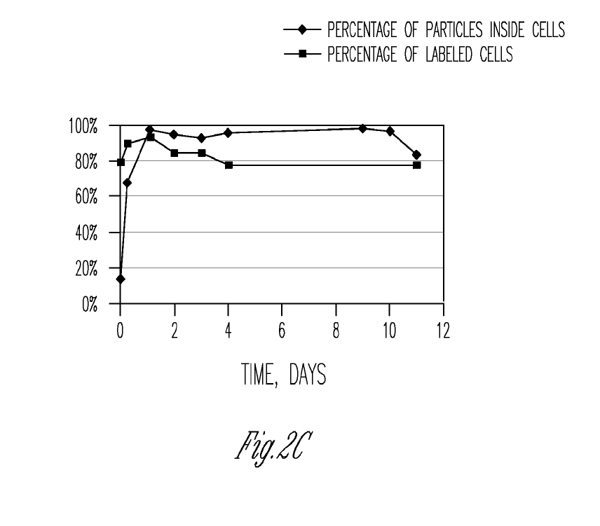

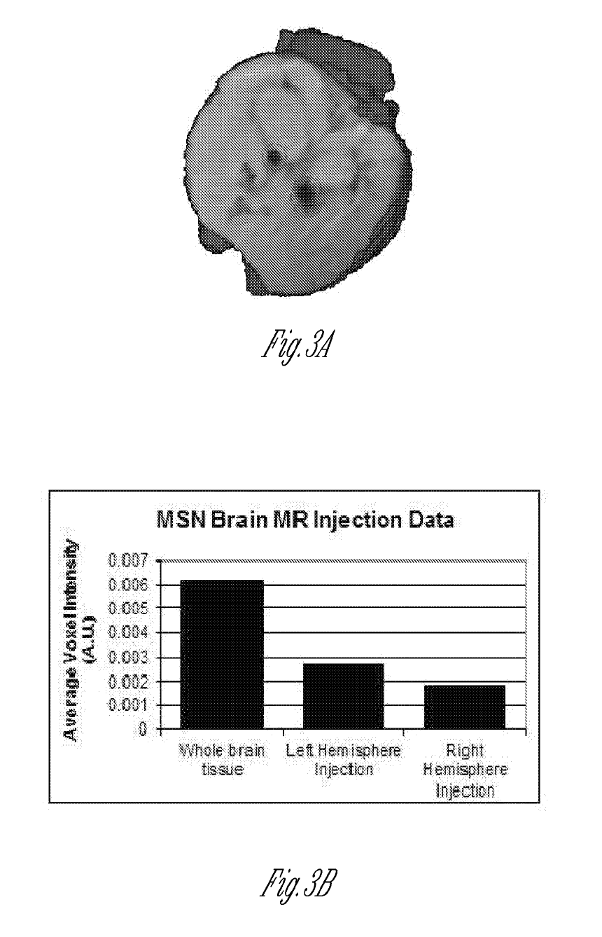

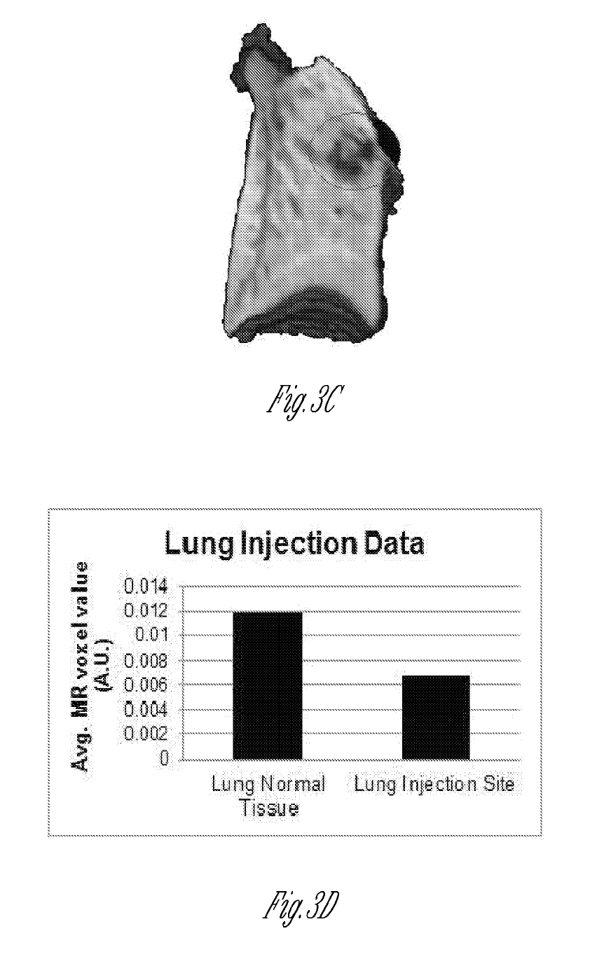

Microinjections of fixed cells labeled with iron oxide-capped FITC-MSN in various mouse organs were made according to the diagrams in FIGS. 3 and 4. FIG. 4 shows the results of long scan T2*-weighted gradient echo MRI sequences, with regions of interest indicated. The injection sites in the brain and lung were isolated by manual segmentation, and their average voxel values compared to those of whole tissue and, in the case of the brain, the lateral ventricles to determine the MR sensitivity to ferrite compared with empty space.

After completing in vitro studies, ex vivo studies were performed on perfusion fixed organs containing labeled stem cells to determine the feasibility of imaging methods. Imaging of several organs (brain, heart and lungs), using several varieties of MSN particles (iron, gold, and bismuth) and either micro-CT, magnetic resonance, or ultrasound imaging modalities, were conducted.

For MR studies, the mouse was anesthetized, perfusion fixed, and injections were made into the fixed organs, which were removed and scanned while immersed in fixative. Not surprisingly, a great deal of contrast using gold and bismuth particles. However, strong contrast was observed using iron oxide MSN particles in brain, heart and lung, with statistically significant observations being made in each tissue.

For ultrasound studies, centrifuge tubes and later agarose phantoms showed the MSN particles gave a high-contrast signal, with Au/FITC-MSN giving about a 30% higher signal than Bi/FITC-MSN. It should be noted, however, that this was at the rather high concentration of 10 mg/mL. When looking at several dilutions of Au/FITC-MSN particles in PBS, we added 20 μL at a time of the stock MSN concentration (200 μg particles) to the 500 μL PBS. The first 2-3 dilutions were not detectable with the naked eye at the time of the experiment; the results seem to show this. Although there is a somewhat large variance in the results, there is a general positive trend which appears to plateau more so than being directly linear. It appears that, by the naked eye, the smallest detectable concentration of particles in PBS is around 1.5 mg/mL, or roughly equal to the concentration of octafluoropropane in mixed Definity®.

The intensity of labeled and unlabeled mesenchymal stem cells was compared, first using cells in suspension in 4% paraformaldehyde, then using cells centrifuged into a pellet. The difference between the labeled and unlabeled cells was undetectable within the large variance of the mixtures. This further confirms that, at concentrations used for intracellular labeling, these particles aren't visible. Another issue arose when scanning the cell pellets: in the highly tapered centrifuge tubes, there is a great deal of artifact arising from the high angled tube walls. Therefore, subsequent experiments involved preparing specimens ahead of time in molten agar, then removing the cooled agar from the plastic dishes.

Overall, using gels to mount specimens was much more effective than centrifuge tubes made of harder plastic that seem to interfere with sound wave propagation more than initially thought. The result of this is that the signal is actually more intense for a smaller concentration of nanomaterial when using agarose. The visual detection threshold seems to be near 200 μg/mL for labeled cells, provided a large enough bolus of cells is used. At this nanoparticle concentration, cell viability can be reduced, so modifications are currently being made to increase the echogenicity of the MSN particles.

Given the fairly high level of detail seen in the cine loops of the scanned heart, a 3D image was generated from the movie. The images may not be truly accurate in all 3-dimensions, as the “z-axis” depends on holding the probe at a constant angle, moving it at a precisely constant speed and in a straight line. Deviations can give rise to distortions in the image. Still, the resulting 3D was quite good, and some loss of detail was due to the many formatting steps. The gross anatomy of the heart can be seen along with what appears to be a hole where the needle was inserted, which is surrounded by slightly hyperintense myocardium, as one would expect if the disclosed contrast agent was injected.

### Example II

Human mesenchymal stem cells (hMSCs) have been the focus of a great deal of regenerative medicine research in recent years, because of its ability to differentiate into many tissue types, including bone, cartilage, tendon, muscle, marrow stroma, and adipose tissue (Bernando et al., 2009; Delorme et al., 2006). Much of the research on these cells is done in vitro on 2-dimensional (2D) tissue culture plastic, while in vivo research has mainly been limited to tagging the cells with a fluorophore and transplanting them into host tissue, followed some time later by sacrifice of the animal and histological analysis. By improving the ability to monitor these cell transplants non-invasively in vivo additional valuable information could be gathered. In addition, the use of fluorophores transfected by viral vector would not translate well to clinical trials, while a more inert nanoparticle detection method would be much more controllable.

An additional trait that may be useful for research or clinical use of hMSCs is to load the cell with a drug such as a growth factor that, upon controlled release, stimulates cell differentiation. As one example, dickkopf-1 (dkk-1) is a Wnt signaling inhibitor that has been shown to regulate the cell cycle of hMSCs (Gregory et al., 2005; Gregory et al., 2003). A scenario in which hMSCs are kept somewhat dormant, then triggered via the dkk-1 mechanism to proliferate only when they reach their destination in specific tissue is certainly a promising one.

MSN uptake by numerous cell types, including HeLa, fibroblasts, breast cancer cells, 3T3-L1, are red blood cells, has been demonstrated. Internalization is controlled by a number of factors including particle morphology and surface chemistry, with endocytosis being the most common pathway. Once internalized, the pathway of the MSN depends significantly on the surface chemistry, with the most favorable result being MSN escape from the endosomal cavity. It may be desirable to release molecules from the mesopores in a controlled fashion in the cytoplasm.

More recently, these particles have been further developed to include novel uncapping mechanisms, such as glucose-triggered release of insulin caps to release both insulin and cyclic AMP (Zhao et al., 2009), as well as electrostatic forces between the reagent and silica (attraction at neutral pH and repulsion at endosomal pH). Additionally, it has been demonstrated that surface treatment of particles with different amounts of functional groups, such as aminopropyl, polyethylene glycol, and carboxyl groups, can fine tune the cell membrane/nanoparticle interaction to achieve a desired amount of cellular uptake or binding (Zhao et al., 2011). Using several bioanalytical techniques, the uptake of these MSN materials by stem cells was detected, and the MSN labeled stem cells or particles alone were tracked in situ using computed tomography (CT), magnetic resonance imaging (MRI), or ultrasound, as described below.

### Materials and Methods

### Synthesis/Characterization of MSN Nanoparticles

The Fe₃O₄ NP and Au NP capped-MSN materials were initially made by the following procedure: First, 1.2 mg fluorescein isothiocyanate was stirred for 20 minutes at room temperature with 10 μL 3-aminopropyltrimethoxysilane (APTMS) in 400 μL anhydrous THF. Next, n-cetyltrimethylammonium bromide (CTAB, 1.0 g, 2.7×10⁻³ mol) was dissolved in 480 mL nanopure water (353 K), made basic with 3.5 mL 2.0 M NaOH. Tetraethyl orthosilicate (TEOS) (5.0 mL, 2.6×10⁻³ mol) was first introduced dropwise, followed by the dropwise addition of the FITC-APTMS/DMF solution. The mixture was stirred for 2 hours at 353 K to give rise to an orange precipitate (as-synthesized FITC-MSN). The solid product was filtered, washed with deionized water and methanol, and dried under vacuum. To remove the surfactant template (CTAB), the as-synthesized FITC-MSN (1.0 g) was refluxed for 18 hours in a solution of 1 mL HCl (37.4%) and 100 mL of methanol, followed by washing with water and methanol.

Fluorescein and bismuth labeled mesoporous silica nanoparticle (FITC-Bi-MSN) materials were synthesized in a similar fashion with the exception of the addition of the dropwise addition of a solution of 1.0 g Bi(NO₃)₃.5H₂O, dissolved in 5.0 mL of acidified nanopure water after the dropwise addition of the FITC labeled ligand.

Gold nanoparticle (Au NP) and iron oxide nanoparticle (Fe₃O₄ NP) caps were synthesized and attached to the MSN via the same procedure described in Giri et al. (2005) and Torney et al. (2007).

### Material Characterization

The materials were characterized by X-ray diffraction in a Rigaku Ultima IV diffractometer, nitrogen sorption analysis in a Micromeritics ASAP 2020 surface area and porosity analyzer using the Brunauer-Emmett-Teller equation to calculate surface area and pore volume and the Barrett-Joyner-Halenda equation to calculate the pore size distribution. The materials were visualized by transmission electron microscopy (TEM) by supporting samples on copper grids in a Tecnai G2 F20 microscope operating at 200 kV.

### Human Mesenchymal Stem Cell Culture

Human fetal MSCs were isolated from 16- to 20-week-old abortuses. The long bones were dissected and transported in 15 mL tubes containing cold Dulbecco's Modified Eagle Medium (DMEM, GIBCO) with 10% heat inactivated fetal bovine serum (fbs, GIBCO). Within an hour of dissection the articular cartilage and periosteum were removed, and the outer bone surface was cleaned by swabbing with Kimwipes® soaked in 70% ethanol. The bone marrow was flushed using a syringe filled with DMEM/10% fbs and a 22 gauge needle, and the cell suspension was divided evenly among 3-60 mm dishes, one of which contained 5 poly-l-lysine-treated 12 mm round glass coverslips.

After 48 hours of incubation, the non-adherent cells were gently rinsed away by repeated pipetting of the culture medium, which was then centrifuged. The adherent cells that remained were given 2 mL fresh DMEM along with 2 mL of the centrifuge supernatant (conditioned medium). The non-adherent cells were resuspended in 4 mL of the remaining conditioned medium and moved to a 25 cm² tissue culture flask for additional studies.

An immunolabeling of STRO-1, an identifier of stromal progenitors (Simmons et al., 1991; Simmons et al., 1994), was also performed on two of the coverslips from the same culture according to standard protocols. Briefly, the coverslips were fixed in a solution of 95% ethanol/5% glacial acetic acid for 5 minutes and rinsed in phosphate buffered saline (PBS, GIBCO). Hybridoma-produced mouse anti human STRO-1 monoclonal antibody (Developmental Studies Hybridoma Bank, University of Iowa) was used at full concentration, and 50 μL was placed on the cells of the coverslip. These were left to react at room temperature for 30 minutes and rinsed with PBS. Next, 50 μL of a 1:50 dilution of goat anti-mouse IgM/FITC was added as the secondary antibody, reacted for 30 more minutes, and rinsed again. The coverslips were mounted on a microscope slide and viewed under 494 nm light. The pattern of STRO-1 labeling was compared with previous studies already published.

### MSN Uptake Kinetics

Uncapped MSNs containing fluorescein (FITC-MSN) as well as MSNs capped with SPIOs (Fe-MSN) were generously provided by Victor S. Y. Lin (Iowa State University). A stock solution of either species of MSN was created by suspending 5 mg powdered MSN in 500 μL PBS. The suspension was sonicated for 30 minutes in a sonicating water bath and a series of dilutions from 10-500 μg/mL were added to 25,000 cells in each well of a 24 well plate. After up to a week of incubation, the maximum dose that did not cause excessive cell death was found to be 125 μg/mL. From that point forward, this was the dosage most commonly used. To study uptake over time, 100 μg MSNs were added to each of the wells of mesenchymal stem cells seeded previously. To view uptake of the particles, the 6 well plate containing FITC-MSN particles was imaged using the Olympus® IX70 fluorescence microscope with green 494 nm filter and with an attached DP70 digital camera and software. Images were obtained at 1 hour, 6 hours, and 26 hours following addition of the particles. After 27 hours, particles that were not engulfed were rinsed from the culture using D-PBS, and more images taken on subsequent days.

Image analysis was done using a MATLAB program. Briefly, the images were normalized to use the entire range of green pixel values from 0-255 with no saturation, then the built-in MATLAB edge detection function was used to isolate single cells. The extracellular area was masked out, and bright green pixels within cells above a user-defined threshold were counted, and these numbers were used to estimate the volume of MSN particles internalized over time.

### Ex Vivo Imaging

Cells were prepared according to the above protocols for isolation and labeling, this time using nanoparticle capped (iron oxide, gold, or bismuth), FITC-loaded MSN particles (Fe/FITC-MSN, Au/FITC-MSN and Bi/FITC-MSN) at a concentration of 125 μg/mL in the growth media. One day after labeling and immediately prior to the experiments, the cells were loosened from the surface of their culture dish using TRYPLEexpress® (GIBCO), suspended in a small volume of phosphate buffered saline (PBS, GIBCO), sampled and counted using Trypan Blue exclusion dye and a hemacytometer. A mouse was then given an intraperitoneal injection of 0.1 mL heparin and anesthetized in a chamber of isoflurane until non-responsive to paw pinching with forceps.

For ex vivo MR imaging of mouse tissue, the chest cavity was opened, and the inferior vena cava (IVC) was severed. A gravity-fed apparatus containing separate volumes of normal saline and 4% paraformaldehyde attached via a ‘Y’ stopcock to a 22-gauge needle was set on a shelf approximately 1.5 meters above the benchtop in order to deliver the fluids at a hydrostatic pressure of about 110 mmHg, or roughly the systolic pressure of a normal mouse. The apparatus was set to inject saline and the needle was inserted into the right ventricle of the mouse to clear the blood from the vasculature. After the blood draining from the IVC ran clear, the apparatus was switched to deliver 4% paraformaldehyde. Perfusion fixation was continued until the mouse's tail curled and then went straight, a sign of muscle fibers cross linking (about 10 minutes of flow). Injections of quantities of Fe/FITC-MSN labeled cells were made into the tissues as noted, as were PBS sham injections and needle sticks only as controls. T2*-weighted pulse echo sequences were used for MR imaging.

For ultrasound imaging, a 1% agarose in PBS solution was made, and held at 50° C. while the other materials were prepared. Gold-capped, FITC-loaded MSN nanoparticles were sonicated for 5-10 seconds, and 20 μL at a concentration of 10 mg/mL was injected into the wall of the left ventricle of a 16 week fetal heart. No visible regurgitation of the injection out of the needle hole could be observed. Ten mL of agarose was poured into a 25 mL beaker onto which the ex vivo heart would be mounted. When it was solid enough to support the weight of the heart, it was mounted in such a way that the ventral heart would be parallel to the flat surface of the agar. An additional 10 mL molten agar was used to cover the heart and it was allowed to set. Next, 5 mL molten agar was poured into each of 4 60 mm dishes. The nanoparticles were added to the agars to make concentrations of 0, 25, 50, and 200 μg/mL, and the stock solution of 10 mg/mL was scanned within its Eppendorf tube as well.

At the ultrasound scanner, the 30 MHz transducer was used. Each prepared agar was removed from its Petri dish, coated with a layer of ultrasound gel, and scanned. For comparison studies, the gain was held constant at 28 dB; all other scanning parameters were kept constant as well. For the heart, several cine loops in both the short axis and long axis were made which ran the length of the entire organ.

For all imaging modalities, the freeware medical image processing program MIPAV was used for image analysis. The isolevel selection tool was used to manually segment volumes of interest (VOIs): in MR heart imaging, the injection sites as well as control volumes for myocardium and paraformaldehyde, and in CT lung imaging, the terminal bronchioles containing labeled cells as well as an unlabeled region in the contralateral lung. For each VOI, MIPAV calculated the mean and standard deviation of intensity value and number of voxels, and these figures were used for pairwise statistical analysis using the t-test for comparison of two means with independent samples and unequal variances.

### Results and Discussion

### MSN Characterization

The structures and surface properties of three different types of MSN were analyzed utilizing a series of different techniques, including transmission electron microscopy (TEM), energy dispersive spectroscopy (EDS), powder X-ray diffraction (XRD), nitrogen sorption, and zeta potential analysis.

Powder XRD analysis confirmed hexagonally arranged mesopores in the diffraction pattern of FITC-Bi-MSN as evident by the intense d₁₀₀, and well resolved d₁₁₀ and d₂₀₀ peaks. Nitrogen sorption analysis of the FITC-Bi-MSN exhibited a Type-IV isotherm, typical of mesoporous materials with a BET surface area of 710 m²g⁻¹. The average pore diameter for FITC-Bi-MSN by BJH calculation is 24 Å.

### MSC Culture Characterization

In order to achieve the ultimate goal of designing a novel in vivo stem cell tracking and drug delivery system, the first step was to confirm the potency of the cultured mesenchymal stem cells to form cells of several lineages. Based on published studies, the cell surface marker STRO-1 has a strong correlation to the cells' differentiability; STRO-1 positive cells are capable of differentiating into tissues as varied as adipogenic, chondrogenic, osteogenic, and myocardial.

Mesenchymal stem cells were isolated from the greater bone marrow population of human fetal long bones by simply separating adherence-dependent from non-adherence-dependent cells after 1-2 days in culture. Then hybridoma-produced monoclonal antibodies to the human STRO-1 marker were used to confirm expression in the adherent cells. It was observed that STRO-1 was localized in the perinuclear regions of the cell, in a similar pattern to that in Simmons et al. (1991). This expression was observed after 5 days in culture, STRO-1 expression diminishes over about 2 weeks in culture.

As further confirmation of the potency of the mesenchymal stem cells, the standard growth medium (DMEM/10% FBS) was switched for medium containing growth factors for adipocytes (StemCell Technologies). After 3-5 days in culture, these cells did change morphology to appear as pre-adipocytes. Compared with the elongated, bipolar morphology of mesenchymal stem cells, pre-adipocytes are more rounded, with several small vacuoles for lipid storage. Mature adipocytes normally have one very large vacuole and stain positive for Oil-Red-O. This observation, along with positive STRO-1 expression, is strong evidence that the isolated cells are indeed stem cells of the bone marrow.

At this optimal dose, early binding of FITC-MSN particles to cell membranes was observed after one hour, with further binding and early endocytosis by 7 hours. At 27 hours, most of the particles appeared to be in the outer regions of cell cytoplasm, and few particles remained outside the cells. At this point the cells were rinsed with phosphate buffered saline to confirm that the particles were indeed bound to the cells, rather than settled loosely on top of them. As the days progressed, the particles were seen to homogenize into fewer, larger compartments and migrate towards the cell nuclei, indicating that they are being compartmentalized, most likely in vesicles. A few dividing cells were observed at 96 hours, showing the equal cytoplasmic division of particles between daughter cells. The same patterns of internalization, compartmentalization and division appeared with the iron oxide- and bismuth-capped MSN particles. It was also observed that a few cells that took up a disproportionately large amount of particles were found to be apoptotic.

### Ex Vivo MRI

The injection sites in the brain and lung were isolated by manual segmentation, and their average voxel values compared to those of whole tissue and, in the case of the brain, the lateral ventricles to determine the MR sensitivity to ferrite compared with empty space.

After completing in vitro studies, ex vivo studies were performed on perfusion fixed organs containing labeled stem cells. Imaging of several organs (brain, heart and lungs), using several varieties of MSN particles (iron, gold, and bismuth) and either micro-CT, magnetic resonance, or ultrasound imaging modalities, were conducted.

For MR studies, the mouse was anesthetized, perfusion fixed, and injections were made into the fixed organs, which were removed and scanned while immersed in fixative. Not surprisingly, there was not a great deal of contrast using gold and bismuth particles. However, strong contrast was observed using iron oxide MSN particles in brain, heart and lung, with statistically significant observations being made in each tissue.

For ultrasound studies, centrifuge tubes and later agarose phantoms showed the MSN particles gave a high-contrast signal, with Au/FITC-MSN giving about a 30% higher signal than Bi/FITC-MSN. It should be noted, however, that this was at the rather high concentration of 10 mg/mL. When looking at several dilutions of Au/FITC-MSN particles in PBS, 20 μL was added at a time of the stock MSN concentration (200 μg particles) to the 500 μL PBS. The first 2-3 dilutions were not detectable with the naked eye. Although there is a somewhat large variance in the results, there is a general positive trend which appears to plateau more so than being directly linear. It appears that, by the naked eye, the smallest detectable concentration of particles in PBS is around 1.5 mg/mL, or roughly equal to the concentration of octafluoropropane in mixed Definity®.

The intensity of labeled and unlabeled mesenchymal stem cells was compared, first using cells in suspension in 4% paraformaldehyde, then using cells centrifuged into a pellet. The difference between the labeled and unlabeled cells was undetectable within the large variance of the mixtures. This further confirms that, at concentrations used for intracellular labeling, these particles aren't visible. Another issue arose when scanning the cell pellets: in the highly tapered centrifuge tubes, there is a great deal of artifact arising from the high angled tube walls. Therefore, subsequent experiments involved preparing specimens ahead of time in molten agar, then removing the cooled agar from the plastic dishes.

Overall, using gels to mount specimens was much more effective than centrifuge tubes made of harder plastic that seem to interfere with sound wave propagation more than initially thought. The result of this is that the signal is actually more intense for a smaller concentration of nanomaterial when using agarose. The visual detection threshold seems to be near 200 μg/mL for labeled cells, provided a large enough bolus of cells is used. At this nanoparticle concentration, cell viability can be reduced, so modifications are currently being made to increase the echogenicity of the MSN particles.

Given the fairly high level of detail seen in the cine loops of the scanned heart, a 3D image was generated from the movie. The images may not be truly accurate in all 3-dimensions, as the “z-axis” depends on holding the probe at a constant angle, moving it at a precisely constant speed and in a straight line. Deviations can give rise to distortions in the image, so this method is more of a “pseudo-tomography.” Some loss of detail in the rendering was due to the many formatting steps. The gross anatomy of the heart could be seen along with what appeared to be a hole where the needle was inserted, which was surrounded by slightly hyperintense myocardium.

### CONCLUSIONS

Mesoporous silica nanoparticles were developed for multimodal, non-invasive tracing of transplanted stem cells. Perhaps the most versatile of the particles is the ferrite capped, FITC-loaded MSN. For in vitro and histological studies, the FITC component allows for fluorescent microscopy, while the ferrous component allows for T2- or T2*-weighted magnetic resonance imaging or Prussian blue staining. The other particles, capped with gold or bismuth, are less effective in MRI but can be seen in computed tomography. All 3 particle types can be seen in ultrasound gel phantoms containing biologically significant amounts.

In addition, the biocompatibility of these particles was confirmed when taken up by STRO-1⁺ human mesenchymal stem cells. For in vivo and clinical studies, the particles may be loaded with a reagent of interest, rather than FITC, further improving their versatility.

### Example III

There are many diseases of the lungs for which computed tomography (CT) imaging is used, including but not limited to emphysema, cystic fibrosis, and lung cancer. In each of these cases, changes in the lung tissue affect its density, which shows up in CT as an anomalous region of interest on the lungs. In some scenarios, such as very early in the disease progression, regions may be small and difficult to visualize without enhanced contrast. In addition, the ability to specifically target a population of cells and deliver a drug of interest or stem cell therapy to counter the disease's progression would be beneficial.

To that end, a mesoporous silicate nanoparticle (MSN) was developed which was loaded with a reagent of interest by simple diffusion and capped using SPIOs. Once internalized, the particles were uncapped through various mechanisms, allowing the controlled release of the product contained therein.

The use of metallic nanoparticles as a label for tracking cells in vivo using CT has not been studied as extensively as MRI; nevertheless, a number of related reports have been published. Hainfield et al. (2006) first injected mice subcutaneously with carcinoma cells, then followed 10 days later with a tail vein injection of 1.9 nm gold nanoparticles at a concentration of 2.7 mg of gold per gram of mouse weight. Using a mammography scanner, the mice were scanned with a photon energy of 22 kVp for a duration of 0.4 s. The nanoparticles enhanced the contrast in the vasculature, particularly highlighting areas of higher blood flow (the subcutaneous tumors). After 60 minutes, the particles were cleared by the kidneys in an equally efficient manner compared with commercially available iodinated contrast agents (Hainfield et al., 2000). A similar study by Rabin et al. (2006) produced equally promising results using 10-40 nm bismuth sulfide nanoparticles. Recently, Cormode et al. (2005) synthesized high density lipoproteins (HDLs) loaded with 5.6 nm gold nanoparticles and observed uptake by macrophages followed by a significant increase in CT contrast compared with unlabeled cells in vitro.

### Methods

### MSN Preparation

Mesoporous silica nanoparticles (MSN) were prepared by the Iowa State lab of Lin/Trewyn using previously described methods. The particles have a mean diameter of 200 nm and a pore size of 5 nm, and were loaded with fluorescein isothiocyanate (FITC) and capped with either iron oxide (Fe₂O₃-FITC-MSN), bismuth (Bi-FITC-MSN), or gold (Au-FITC-MSN). Prior to use, a stock suspension was prepared at a concentration of 10 mg/mL and sonicated using an ultrasonic probe to reduce particle agglomeration.

### Cell Culture

Human fetal mesenchymal stem cells (MSCs) were prepared by bone marrow aspiration and selected by adherence to tissue culture plastic. At 5 days in vitro in a parallel culture on 12 mm poly-L-lysine-coated glass coverslips, immunofluorescence was performed to confirm expression of STRO-1, an important MSC marker. After the second passage, 3 confluent 100 mm dishes of cells were labeled with Fe₂O₃-FITC-MSN, Bi-FITC-MSN, or Au-FITC-MSN at a concentration of 125 μg/mL. To view uptake of the particles, the 6 well plate containing FITC-MSN particles was imaged using the Olympus® IX70 fluorescence microscope with green 494 nm filter and with an attached DP70 digital camera and software. Images were obtained at 1 hour, 6 hours, and 26 hours following addition of the particles. After 27 hours, particles that were not engulfed were rinsed from the culture using D-PBS, and more images taken on subsequent days.

### Imaging/Image Processing

For mouse lung micro-CT imaging, Balb/c mice between 20-22 g weight were initially sedated with 3% isoflurane, followed by intraperitoneal injection of ketamine 50 mg/kg. Anasthesia was confirmed when the mouse became non-responsive to tail-pinch stimulus. Next, the trachea was exposed, partially cut, and cannulated with a flexible 22 gauge Luer-lok cannula. Through the cannula, 1.7×10⁶ cells fixed in 20 μL 4% paraformaldehyde and labeled with one type of MSN particles were delivered to one of the lungs. The lungs were then inflated by connecting the cannula to a source of air pressure and the chest cavity was opened to perform perfusion fixation. A needle was inserted in the left ventricle and the inferior vena cava was cut, and room temperature phosphate buffered saline (PBS) was delivered using a gravity fed apparatus. After the blood was cleared, the apparatus was used to deliver 4% paraformaldehyde at approximately 1 mL/minute for a minimum of 10 minutes. The heart/lungs were removed and dried in a 55° C. drying oven for a minimum of 3 days with constant application of intratracheal pressure.

Once dried, the heart and lungs were scanned using an Imtek Micro-CAT II scanner (Siemens Pre-Clinical Solutions). In initial experiments, various scan parameters were used until optimal parameters were found: 50 kVp source voltage, 400 μA source current, 400 ms exposure, and 720 projections over 270 degrees of rotation. The reconstructed images were 480×479×640 pixels with a 28 μm isotropic voxel size, making the field of view 13.44×13.41×17.92 mm.

For post-processing and quantitative analysis of images, the freeware medical image processing program MIPAV was used. Using manual segmentation and the isolevel volume of interest (VOI) selection tool, the heart was segmented and removed, and the portion of lung that appeared to be hyperintense was selected, along with a control region in the contralateral lung. For each VOI, MIPAV calculated the mean and standard deviation of intensity value and number of voxels, and these values were used for pairwise statistical analysis using the t-test for comparison of two means with independent samples and unequal variances:

$$t = \frac{( {\overline{x}}_{1} - {\overline{x}}_{2} ) - ( {μ}_{1} - {μ}_{2} )}{\sqrt{( {s}_{1}^{2} / {n}_{1} ) + ( {s}_{2}^{2} / {n}_{2} )}} ; v = \frac{{[ \frac{{s}_{1}^{2}}{{n}_{1}} + \frac{{s}_{2}^{2}}{{n}_{2}} ]}^{2}}{[ \frac{{( {s}_{1}^{2} / {n}_{1} )}^{2}}{{n}_{1} - 1} + \frac{{( {s}_{2}^{2} / {n}_{2} )}^{2}}{{n}_{2} - 1} ]}$$

where n₁ and n₂ are the number of voxels in each VOI, s₁² and s₂² are their respective standard deviations, μ₁ and μ₂ are their means and ν is the degrees of freedom used in reference to the statistical lookup table.

### Results

### Cell Culture/Particle Characterization

Human mesenchymal stem cells were isolated from aspiration of bone marrow, and isolated by their adhesion to tissue culture plastic. At 5 days in vitro, these cells were labeled with STRO-1 antibody to confirm their progenitor phenotype. The pattern of perinuclear, punctiform labeling of STRO-1 on human mesenchymal stem cells isolated from bone marrow aspirates is typical of patterns previously shown (Simmons and Torok-Storb, 1991).

Next, uptake of the particles by these cells was confirmed through fluorescence microscopy. Early binding of FITC-MSN particles to cell membranes was observed after one hour, with further binding and early endocytosis by 7 hours. As the days progressed, the particles were seen to homogenize into fewer, larger compartments and migrate towards the cell nuclei, likely indicating that they are being compartmentalized into vesicles. A few dividing cells were observed throughout the experiment, showing the equal cytoplasmic division of particles between daughter cells. The same patterns of internalization, compartmentalization and division appeared with the Fe-MSN particles. The cells grew in a manner similar to unlabeled cells, doubling in number about every 3 to 4 days until reaching contact inhibition in about 10 days.

### CT Imaging

In early experiments involving micro-CT of the lung with Fe₂O₃-FITC-MSN particles, the cannula slipped, allowing the lung to become deflated. Although attempts to reinflate the lungs were made prior to drying, once imaged, the effects of deflation could be seen. The bronchioles were somewhat compressed, causing areas of higher density and masking any signal that may have otherwise been observed. After improving suturing methods and optimizing scan parameters, labeled cells with each nanoparticle type were administered to one mouse each, and the results are shown below.

When thresholding is used to isolate the airway tree in lungs injected with Fe₂O₃-FITC-MSN labeled cells, there remained a region in the apex of the left lung outside the local bounds of the airway. It appears the labeled cells were placed in the bronchiole LMB2. This was identified as the volume of interest and its intensity (in 16-bit greyscale values) was compared to a region from the contralateral lung. The volume of interest had a mean value of −696±171 and the contralateral region had a mean value of −942±40.

In lungs injected with Au-FITC-MSN labeled cells, the volume of interest appears to be the accessory lobe of the right lung, fed by bronchiole AcRMB3. The volume of interest had a mean value of −711±145 and the contralateral region had a mean value of −894±62.

In lungs injected with Bi-FITC-MSN labeled cells, the volume of interest appears to be the middle lobe of the right lung, fed by bronchiole MiRMB3. The volume of interest had a mean value of −843±87 and the contralateral region had a mean value of −954±38.

The control volumes have a lower intensity mean and generally narrower peak with low variance, whereas the labeled volumes of interest have a broad peak and slightly higher mean and variance. Each label volume was statistically compared to its control volume and the result was a statistically significant difference with p<0.01.

### Discussion

In order to achieve the ultimate goal of designing a novel in vivo stem cell tracking and drug delivery system, the first step was to confirm the potency of cultured mesenchymal stem cells to form cells of several lineages. Based on published studies, the cell surface marker STRO-1 has the strongest correlation to the cells' differentiability than any other marker. STRO-1 positive cells are capable of differentiating into tissues as varied as adipogenic, chondrogenic, osteogenic, and myocardial.

Mesenchymal stem cells were isolated from the greater bone marrow population of human fetal long bones by simply separating adherence-dependent from non-adherence-dependent cells after 1-2 days in culture. Next, hybridoma-produced monoclonal antibodies to the human STRO-1 marker were used to confirm expression in the adherent cells. Because immunological staining techniques can be sensitive to non-specific binding and other false positives, it is important to observe not just the presence or absence of signal, but to examine the pattern of expression observed, and compare that using published results as a sort of positive control. In this case, it was observed that STRO-1 was localized in the perinuclear regions of the cell, in a punctiform pattern similar pattern to published data. This expression was observed after 5 days in culture.

Initially, the optimal dose of MSN was measured. In a 24 well culture plate, 2.5×10⁴ mesenchymal stem cells were seeded per well, and the MSN doses (in μg/mL) were 10, 25, 50, 125, 250, and 500. The maximum dose that did not cause an excessive observable toxicity to the cells was 125 μg/mL. For each new variety of MSN particle this dosage experiment was repeated, observing a maximum safe dose of 125 μg/mL each time.

Given that result, each subsequent experiment was performed using 125 μg/mL as a standard dose for all MSN particles. Therefore, a new culture was prepared, using a slightly sparser initial cell population so that it may be observed over a longer time course. After adding the particles, the cells were observed several times throughout the first day, then daily thereafter. Within hours, the particles could be seen aligning with the cell membranes, and lightly tapping the culture plate showed that the particles did not move from this position. They appeared to be effectively stuck to the cell membranes, rather than loosely associated. The following day, nearly all of the particles were inside the cells, and on subsequent days, the clusters of intracellular particles appeared to decrease in number but increase in size. In addition, the majority of the particles seemed to be moved towards the nuclei of the cells as the days progressed.

For micro-CT studies involving the lung, a method was employed for perfusion fixation of the mouse that allowed the loading of particles into the airway tree of the lung without collapsing the delicate lung tissue upon dissection. After performing a tracheotomy, injecting the cells intratracheally, and fixing the lungs under pressure, we observed hyperintense regions in the lung tissue at the most inferior terminal bronchi of the lung containing labeled stem cells. Measuring the grayscale values of the regions containing labeled stem cells and comparing with normal regions of the contralateral lung, a statistically significant change in signal was found for each of the particles, with the greatest degree of significance in gold particles.

In CT imaging, the signal-to-noise ratio is a function of the number of x-ray photons that reach the detector for each pixel. This is affected by the x-ray source voltage, the properties of the collimator which reduces noise from scattered photons, and the spatial resolution. The source voltage remains the same regardless of whether the system is a micro-CT or larger clinical CT scanner. The properties of the collimator are unknown, but are assumed to be similar for both scanner types. The biggest difference between the scanner types is the spatial resolution, which can be under 50 μm for micro-CT, and around 0.5 mm for clinical scanners. Therefore, for a volume of labeled cells to be detected within a voxel that is 10 times larger, the volume should be 10 times larger as well. It is reasonable then to assume that a mass of 17 million cells would be detected in the lungs of a human subject.

Because the scans for analysis used the same parameters, it was expected that equivalent values for normal, unlabeled lung tissue would be observed. Within 1 standard deviation, this appears to be the case. Because of their increasing atomic weight, bismuth was expected to have the greatest difference in intensity, followed by gold, then iron. However, gold and iron had almost identical histograms, and the intensity of bismuth was actually lower. This may be due in part to the particles dispersing more in the bismuth and gold specimens.

After looking at the results of all 3 particle types, it appears that each lung has a region of some volume which has a greater intensity than its surrounding tissue and its contralateral lung. This, in addition to careful histology of labeled and non-labeled, regions can provide strong evidence that the intensity change is due to the particle injection.

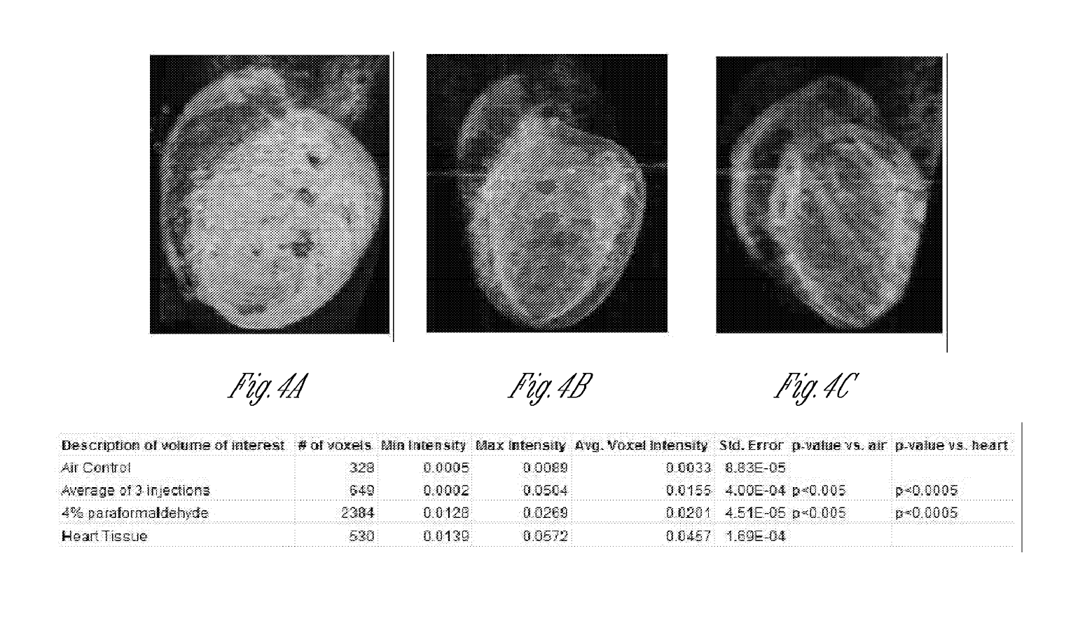

When uncoated bismuth is injected (e.g., about 1.7 mg), it has been reported to be toxic even at doses as low as 8 mM. For a review of bismuth applications in medicine, including a summary of toxicity, see Briand and Burford, (Chem. Rev., 99:2601 (1999)). A Bi-FITC-MSN particle which has improved nanoparticle morphology control was prepared. The particles incorporate the bismuth moiety in the inner side of the MSN which lessens bismuth exposure. A schematic of this strategy is shown in FIG. 5B. CT imaging of such a particle shows that a clear signal was detected with no appreciable toxicity to adjacent cellular material. These particles showed good biocompatibility at the typical dose of 125 μg/mL. Because of the low toxicity and the high contrast signal obtained with these nanoparticles, even a higher concentration of particles may be employed, e.g., the range employed above is about 15 fold lower than a bismuth injection, e.g., as described above, and so is safer and more effective.

### Example IV

### Materials and Methods

### Preparation of In Vitro and Ex Vivo Phantoms

A preparation of 2% agarose in PBS was made and held at 50° C. while the other materials were prepared. Nanoparticles (Au-FITC-MSN, PEG-Gd₂O₃-MSN, or PEG-coated F₃-FITC-Gd₂O₃— MSN) were sonicated for 5-10 seconds and 20 μL at a concentration of 10 mg/mL was injected into the wall of the left ventricle of a 16 week fetal heart. No visible regurgitation of the injection out of the needle hole could be observed. Next, 5 mL molten agar was poured into each of 7 35 mm dishes as well as about 10 mL into a 25 mL beaker into which the heart would be mounted. The nanoparticles were added to the agars to make concentrations of 0, 100, 250, 500, and 1000 μg/mL.

At the ultrasound scanner, the 30 MHz transducer was used. Each prepared agar was carefully removed from its Petri dish, coated with a layer of ultrasound gel, and scanned. For comparison studies, the gain was held constant at 28 dB; all other scanning parameters were kept constant as well. For the heart, several cine loops in both the short axis and long axis were made which ran the length of the entire organ. Additionally, a cine loop was used to record injection of PEG-F₃-FITC-Gd₂O₃-MSN into the right ventricle of an ex vivo heart in real time.

### Image Processing

Image processing was performed using a combination of freeware processing programs ImageJ and MIPAV. For each image, the volume of interest (VOI) showing contrast within the Eppendorf tube was manually selected using the freehand tool. A histogram containing the pixel count, mean grayscale value and standard deviation was obtained. A 3D rendering of the ex vivo heart was generated using cine loops which scanned through the heart on the short axis and output in the form of a compressed AVI file. In ImageJ, the AVI files were loaded and each frame was saved as a TIF image stack, which can be analyzed in both ImageJ and MIPAV.

### Results and Discussion

### In Vitro Experiments

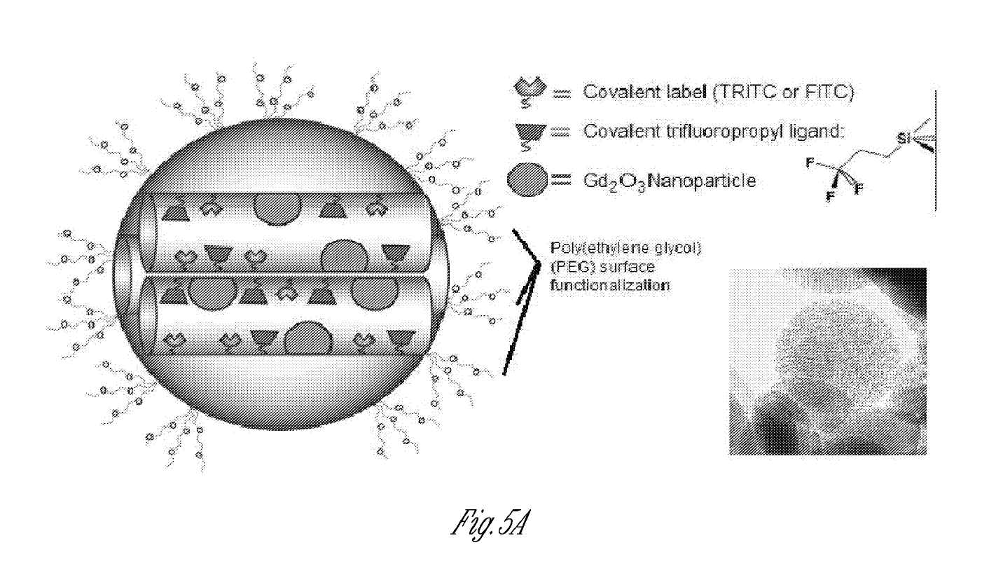

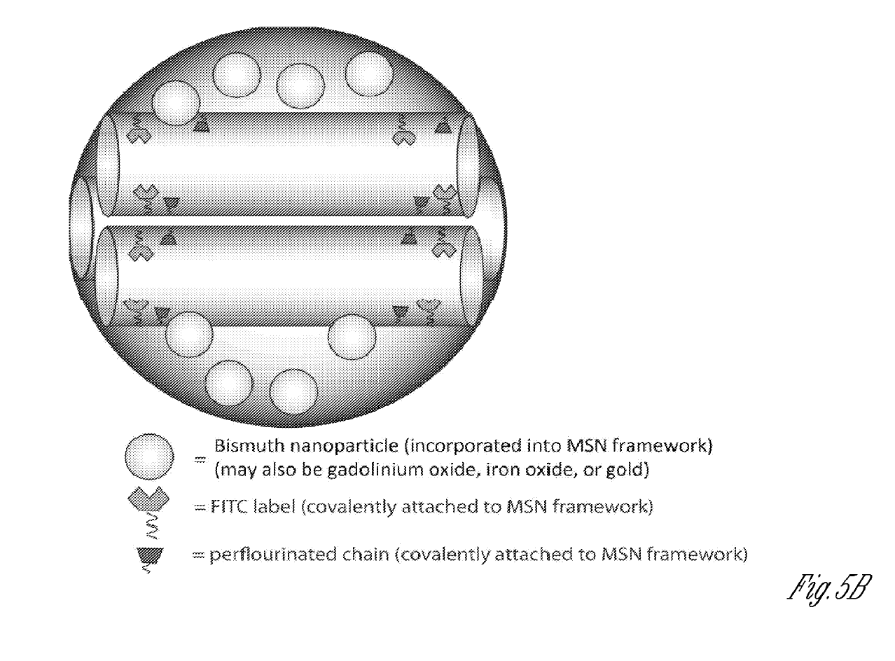

In the agarose disk samples containing PEG-functionalized F₃-FITC-Gd₂O₃-MSN, even at concentrations as low as 62.5 μg/mL, a difference can be seen compared with the PBS control (FIG. 6). However, the PEG-Gd₂O₃-MSN particles were much less echogenic. Essentially the only difference between these particles is in the inclusion of trifluoropropyl (—CH₂—CH₂—CF₃) moiety, which appears to be the source of nearly all the observed signal.

In terms of absolute grayscale intensity, the signal generated by the MSN is significantly smaller compared with the various dilutions of Definity®, which seemed to reach a plateau grayscale value even at very small concentrations. By comparison, our F₃-FITC-Gd₂O₃-MSN nanoparticles appear to follow a more linear trend however, which may be valuable in quantifying the amount of material in a given volume.

While the contrast generated by the MSN is lower than that of the commercially available Definity®, our particles can be detected and, given sufficient accumulation in a small area, can be nearly as intense. Clinical contrast agents are typically 1-5 μm in diameter, which allows them to flow through the smallest capillaries while still generating a strong echo within clinical scanners, many of which have a relatively low spatial resolution. The use of nanoscale contrast agents in ultrasound imaging is only rarely done in research at this time, but there exist some niches in which nanoscale ultrasound contrast may be useful. While micron-scale agents are nearly the size of a cell, nanoscale materials are small enough to accumulate within cells. This is useful in times when a bolus of stem cells may be labelled with contrast agents such that their engraftment may be tracked non-invasively, or if a particle were designed for targeted, intracellular drug delivery. Accumulation of a large number of targeted nanoparticles in a region of tissue may also be detected via ultrasound. Three different types of labelled mesoporous silica nanoparticle materials were successfully synthesized and characterized by standard techniques.

### Ex Vivo Echocardiography Validation

Prior to development of PEG-F₃-FITC-Gd₂O₃-MSN, early ex vivo experiments involved injection of Au-FITC-MSN particles. In the first such experiment, 200 μg Au-FITC-MSN in 20 μL saline were injected into the left ventricular wall of a paraformaldehyde-fixed ex vivo heart, then immersing the heart in a 15 mL centrifuge tube containing 4% paraformaldehyde and scanning the specimen through the wall of the tube. Although a small hyperintense region at the site of injection could be observed, this proved to be a suboptimal setup for measuring signal, as the walls of the tube were a large source of artefact.

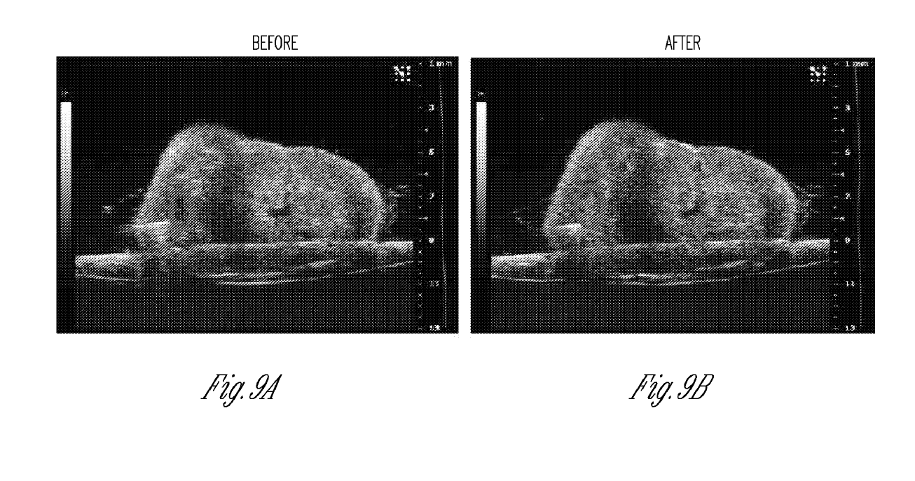

Subsequently, the heart was mounted in 2% agarose and rescanned. The noise was greatly reduced (FIG. 9) and the agarose more closely mimics the thoracic cavity through which the in vivo heart would be scanned. With greater detail, the ultrasound transducer was moved along the short axis of the heart to generate a cine loop with frames that were essentially 2D tomographic slices which make up a 3D image. Because the transducer may not have moved at a uniform speed, each slice may not represent a precise thickness, thus providing a “pseudo-tomography.” When viewed as a 3D rendering, the VOI seemed to be a dark needle track surrounded by slightly hyperintense tissue as a result of infiltration of MSN particles.

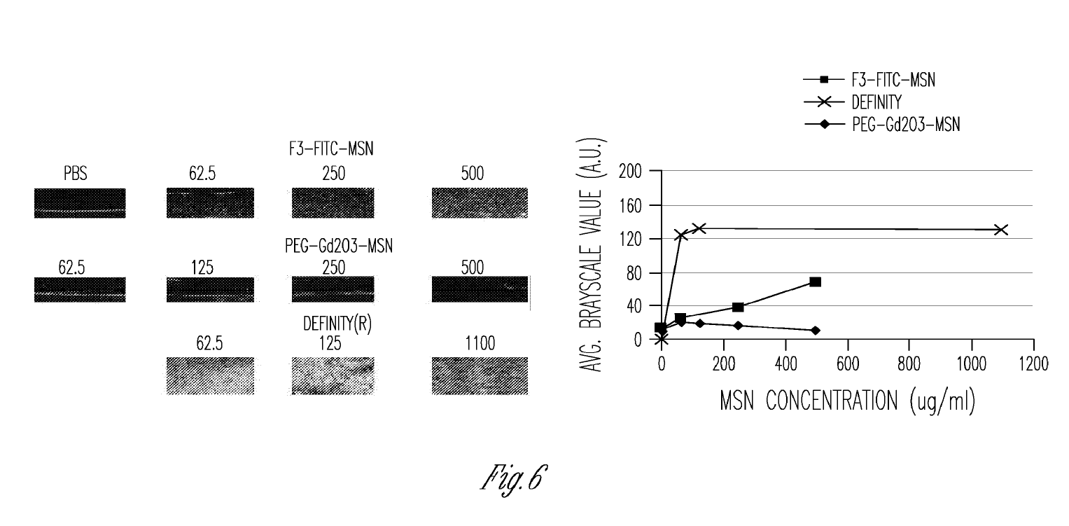

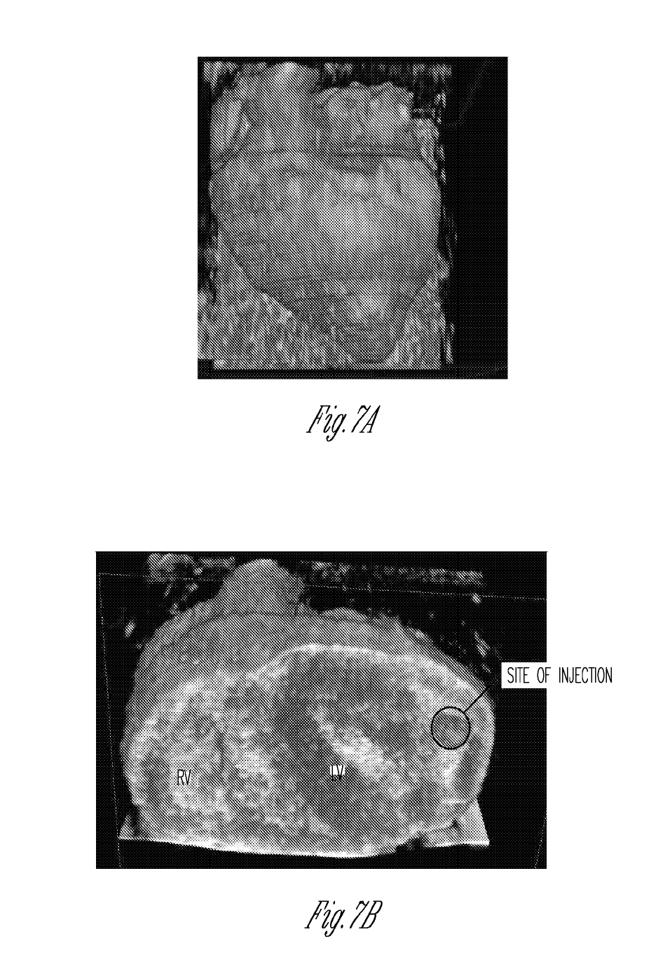

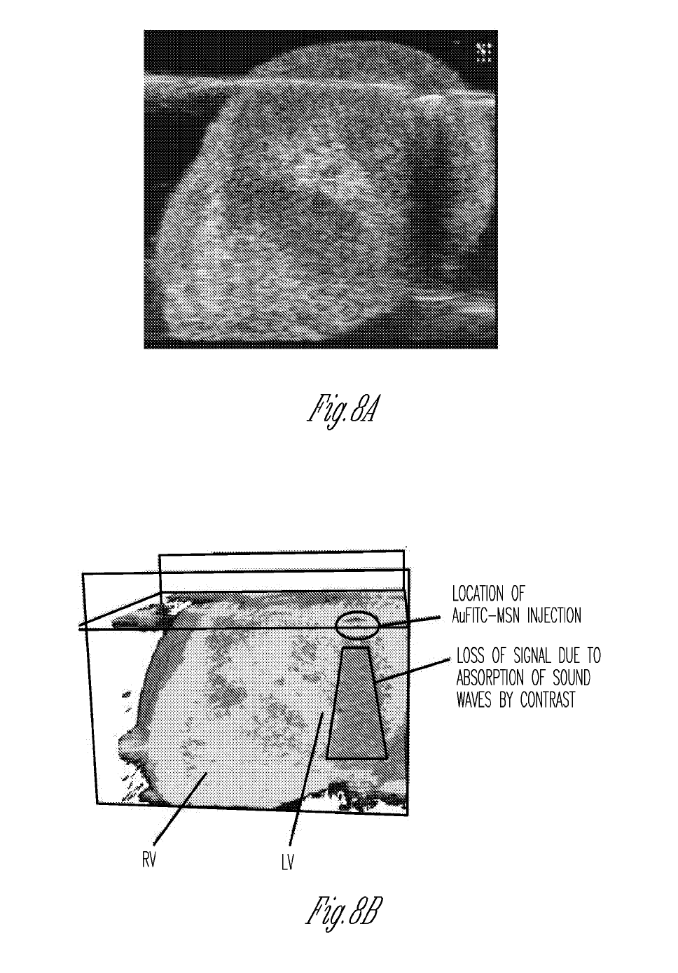

In a second heart, also injected with 200 μg Au-FITC-MSN in 20 μL saline, a more prototypical signal could be observed (FIG. 7). Rather than diffusing, as the previous injection appeared to have done, the contrast remained in a compact, hyperintense bolus in the myocardial wall, and the tissue further from the transducer was shadowed as a result of the obstruction of sound waves by the material.

Finally, the contrast was observed in real-time. A previously non-labeled ex vivo heart was mounted in 2% agarose and scanned in the short axis at 30 MHz using the cine loop to collect 100 frames of real-time data at 20 frames per second. A pipetting device was fitted with a 27 gauge hypodermic needle for precise delivery of 20 μL volume of F₃-FITC-Gd₂O₃-MSN particles through the agar into the right ventricular chamber. Upon locating the needle in the ultrasound monitor, the cine loop was started and the particles were deployed. The ejection of contrast agent from the needle could be observed, with frames immediately before and after injection showing the change in contrast of the ventricular chamber (FIG. 9).

This result shows the particles can be used in the same manner as ultrasound contrast, such as Definity®, in real-time imaging strategies. As with the in vitro studies, the observed signal is not as large as that for clinical contrast agents, but there exist other features which may make these materials favorable in certain conditions. Clinical contrast agents are somewhat unstable-from the circulation, they reach the lungs and the encapsulated high molecular weight gases readily diffuse into the airways and are quickly exhaled. MSN-based ultrasound contrast agents would have more long-term stability for the potential for imaging over multiple days. When they are cleared from the bloodstream, they accumulate in the filter organs (lung, liver, kidneys) until they can be excreted, but they may also be functionalized with receptor ligands for tissue-specific contrast enhancement.

### CONCLUSIONS

A family of novel mesoporous silica nanoparticles was developed for multimodal, non-invasive tracing of transplanted stem cells. Gold capped, FITC-loaded MSN (Au-FITC-MSN) can be used as a contrast in x-ray computed tomography (CT), and PEG-coated, trifluoropropyl functionalized gadolinium oxide nanoparticles (PEG-F₃—Gd₂O₃-FITC-MSN) can be used in T1-, T2-, or T2*-weighted magnetic resonance imaging (MRI) applications. With the presence of FITC, both of those can be used for fluorescent microscopy, and as a potential ultrasound contrast agent.

### REFERENCES

Bara et al., J. Am. Soc. Echocardio., 19:563 (2006).
Bernardo et al., W. E. Annals of the New York Academy of Sciences, 1176:101 (2009).
Briand and Burford, Chem. Rev., 99:2601 (1999).
Bridot et al., J. Am. Chem. Soc., 129:5076 (2007).
Casciaro et al., Invest. Radiology, 45:715 (2010).
Chelliah and Senior, Curr. Card. Rep., 11:216 (2009).
Cormode et al., Nano Lett., 8:3715 (2008).
Delorme et al., Regenerative medicine, 1:497 (2006).
Dubois et al., Nat. Biotech., 29:1011 (2011).
Englebrecht et al., Rofo, 165:24 (1996).
Garcia-Pacheco et al., Mol. Hum. Reprod., 7:1151 (2001).
Giri et al., Angewandte Chemie (International ed. in English), 44:5038 (2005).
Gregory et al., J. Biol. Chem., 278:28067 (2003).
Gregory et al., J. Biol. Chem., 280:2309 (2005).
Gronthos et al., Blood, 84:4164 (1994).
Gruenhagen et al., Applied Spectroscopy, 59:424 (2005).
Hainfeld et al., Br. J. Radiol., 79:248 (2006).
Hsiao et al., Small (Weinheim an der Bergstrasse, Germany), 4:1445. (2008)
Kwon et al., App. Surf. Sci., 254:4732 (2008).
Lai et al., J. Am. Chem. Soc., 125:4451 (2003).
Lauffer, Chem. Rev., 87:901 (1987).
Liang et al., IEEE Engineering in Medicine and, Biology Society In Principles of magnetic resonance imaging: a signal processing perspective/Zhi-Pei Lian; SPIE Optical Engineering Press; IEEE Press: Bellingham, Wash.: New York, N.Y.:, 2000
Mallidi et al., Conference Proceedings: Annual International Conference of the IEEE Engineering in Medicine and Biology Society. IEEE Engineering in Medicine and Biology Society. Conference, 2009, 2009, 6338-40.
Rabin et al., Nat. Mater., 5:118 (2006).
Radu et al., J. Am. Chem. Soc., 126:13216 (2004).
Simmons et al., Blood, 78:55 (1991).
Simmons et al., Prog. Clin. and Biol. Res., 389:271 (1994).
Slowing et al., J. Am. Chem. Soc., 128:14792 (2006).
Slowing et al., J. Am. Chem. Soc., 129:8845 (2007).
Torney et al., Nat. Nanotechnol., 2:295 (2007).
Uosaki et al., PLoS One, 6:e23657, epub 18 Aug. 2011.
Webster and Clark, In Medical instrumentation: application and design/John G. Webster, editor; contributing; Wiley: New York: 1998; Vol. 3rd ed.
Yang et al., Biomaterials, 31:854 (2010).
Yu et al., Chem. Rev., 99: 2353 (1999).
Zhao et al., ACS Nano, 5:1366 (2011).
Zhao et al., J. Am. Chem. Soc., 131:8398 (2009).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method for sequential imaging in a mammal, comprising:
a) introducing to a mammal a composition comprising mesoporous silica nanoparticles (MSNs) comprising a lanthanide, a fluorophore and an agent detectable by ultrasound;
b) applying ultrasound and a magnetic field to the mammal and recording ultrasound and magnetic resonance images that include the MSNs; and
c) detecting the presence, location or amount of the MSNs in the mammal.

2. A method to image diagnostic or therapeutic cells in a mammal, comprising: a) introducing to a mammal a composition comprising mammalian cells comprising mesoporous silica nanoparticles (MSNs) comprising a lanthanide, a fluorophore and an agent detectable by ultrasound;
b) applying ultrasound and/or a magnetic field to the mammal and recording ultrasound and/or magnetic resonance images that include the MSNs; and
c) detecting the presence, location or amount of the MSNs in the mammal.

3. The method of claim 1 wherein the MSNs are about 1 nm to about 50 nm in diameter.

4. The method of claim 1 wherein the MSNs are less than about 200 nm in diameter.

5. The method of claim 1 wherein the MSNs comprise pores about 1 nm to about 10 nm in diameter.

6. The method of claim 1 wherein the MSNs further comprise a targeting agent.

7. The method of claim 6 wherein the agent is an antibody or an antigen binding portion thereof.

8. The method of claim 1 wherein the images are of a heart, lung, kidney, liver, pancreas, bladder, ovary, uterus, or brain.

9. The method of claim 1 wherein the composition is injected into the mammal.

10. The method of claim 9 wherein the composition is subcutaneously, intradermally or intravascularly injected into the mammal.

11. The method of claim 1 wherein the mammal is a human.

12. The method of claim 2 wherein the cells are mesenchymal stem cells.

13. The method of claim 2 wherein the cells are pluripotent stem cells.

14. The method of claim 2 wherein the cells are embryonic stem cells, umbilical cord cells, bone marrow cells, peripheral blood cells, adult-derived stem or progenitor cells, tissue-derived stem or progenitor cells, or multipotent adult progentitor cells (MAPC) cells.

15. The method of claim 2 wherein the cells are allogeneic cells.

16. The method of claim 1 wherein the lanthanide is gadolinium.

17. The method of claim 16 wherein the gadolinium in the MSNs is a nanoparticle.

18. The method of claim 1 wherein the ultrasound image is recorded before the magnetic resonance image.

19. The method of claim 1 wherein the agent comprises trifluoropropyl groups.

20. A composition comprising mesoporous silica nanoparticles (MSNs) comprising gadolinium oxide nanoparticles, a fluorophore and an agent detectable by ultrasound.

21. The composition of claim 20 wherein the agent comprises trifluoropropyl groups.

22. Use of a composition comprising mesoporous silica nanoparticles (MSNs) comprising gadolinium oxide nanoparticles, a fluorophore and an agent detectable by ultrasound in the preparation of a medicament for imaging.
